SEQUENCE LISTING (4 sequences)

SEQ ID NO: 1 - protein, 512 aa, Marinobacter algicola strain DG893
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKAA

SEQ ID NO: 2 - protein, 513 aa, Marinobacter aquaeolei
MAIQQVHHADTSSSKVLGQLRGKRVLITGTTGFLGKVVLERLIRAVPDIGAIYLLIRGNKRHPDARSRFLEEIATSSVFDRLREADSEGFDAFLEERIHCVTGEVTEAGFGIGQEDYRKLATELDAVINSAASVNFREELDKALAINTLCLRNIAGMVDLNPKLAVLQVSTCYVNGMNSGQVTESVIKPAGEAVPRSPDGFYEIEELVRLLQDKIEDVQARYSGKVLERKLVDLGIREANRYGWSDTYTFTKWLGEQLLMKALNGRTLTILRPSIIESALEEPAPGWIEGVKVADAIILAYAREKVTLFPGKRSGIIDVIPVDLVANSIILSLAEALGEPGRRRIYQCCSGGGNPISLGEFIDHLMAESKANYAAYDHLFYRQPSKPFLAVNRALFDLVISGVRLPLSLTDRVLKLLGNSRDLKMLRNLDTTQSLATIFGFYTAPDYIFRNDELMALANRMGEVDKGLFPVDARLIDWELYLRKIHLAGLNRYALKERKVYSLKTARQRKKAA

SEQ ID NO: 3 - protein, 514 aa, Oceanobacter sp. strain RED65
MSQYSAFSVSQSLKGKHIFLTGVTGFLGKAILEKLLYSVPQLAQIHILVRGGKVSAKKRFQHDILGSSIFERLKEQHGEHFEEWVQSKINLVEGELTQPMFDLPSAEFAGLANQLDLIINSAASVNFRENLEKALNINTLCLNNIIALAQYNVAAQTPVMQISTCYVNGFNKGQINEEVVGPASGLIPQLSQDCYDIDSVFKRVHSQIEQVKKRKTDIEQQEQALIKLGIKTSQHFGWNDTYTFTKWLGEQLLIQKLGKQSLTILRPSIIESAVREPAPGWVEGVKVADALIYAYAKGRVSIFPGRDEGILDVIPVDLVANAAALSAAQLMESNQQTGYRIYQCCSGSRNPIKLKEFIRHIQNVAQARYQEWPKLFADKPQEAFKTVSPKRFKLYMSGFTAITWAKTIIGRVFGSNAASQHMLKAKTTASLANIFGFYTAPNYRFSSQKLEQLVKQFDTTEQRLYDIRADHFDWKYYLQEVHMDGLHKYALADRQELKPKHVKKRKRETIRQAA

SEQ ID NO: 4 - protein, 460 aa, Bombyx mori
MSHNGTLDEHYQTVREFYDGKSVFITGATGFLGKAYVEKLAYSCPGIVSIYILIRDKKGSNTEERMRKYLDQPIFSRIKYEHPEYFKKIIPISGDITAPKLGLCDEERNILINEVSIVIHSAASVKLNDHLKFTLNTNVGGTMKVLELVKEMKNLAMFVYVSTAYSNTSQRILEEKLYPQSLNLNEIQKFAEEHYILGKDNDEMIKFIGNHPNTYAYTKALAENLVAEEHGEIPTIIIRPSIITASAEEPVRGFVDSWSGATAMAAFALKGWNNIMYSTGEENIDLIPLDYVVNLTLVAIAKYKPTKEVTVYHVTTSDLNPISIRRIFIKLSEFASKNPTSNAAPFAATTLLTKQKPLIKLVTFLMQTTPAFLADLWMKTQRKEAKFVKQHNLVVRSRDQLEFFTSQSWLLRCERARVLSAALSDSDRAVFRCDPSTIDWDQYLPIYFEGINKHLFKNKL

(19) United States
(12) Patent Application Publication
(10) Pub. No.: US 2015/0017698 A1
(43) Pub. Date: Jan. 15, 2015

(54) RECOMBINANT HOST CELLS AND PROCESSES FOR PRODUCING 1,3-BUTADIENE THROUGH A 5-HYDROXYPENT-3-ENOATE INTERMEDIATE

(71) Applicant: Codexis, Inc., Redwood City, CA (US)

(72) Inventors: Gregory A. Cope, Menlo Park, CA (US); Louis Clark, San Francisco, CA (US)

(73) Assignee: Codexis, Inc. a corporation

(21) Appl. No.: 14/381,137

(22) PCT Filed: Feb. 26, 2013
(86) PCT No.: PCT/US2013/027730

§ 371 Date: Aug. 26, 2014

Related U.S. Application Data
(60) Provisional application No. 61/606,228, filed on Mar. 2, 2012.

(51) Int. Cl. C12P 5/02; C12N 15/76; C12N 15/81; C12N 15/70; C12N 15/75

(52) U.S. Cl. CPC C12P 5/026; C12N 15/70; C12N 15/75; C12N 15/81; C12N 15/815; C12N 15/76
USPC 435/167; 435/252.33; 435/252.31; 435/254.21; 435/254.2; 435/252.35; 435/471

(57) ABSTRACT

The present disclosure relates to recombinant host cells comprising one or more recombinant polynucleotides encoding enzymes in select pathways that provide the ability to use the cells to produce 1,3-butadiene. The present disclosure also provides methods of manufacturing the recombinant host cells, and methods for the use of the cells to produce 1,3-butadiene. The methods utilize recombinant host cells that comprise an engineered pathway of enzymes that provides for the conversion of naturally occurring intermediate crotonyl-CoA (or -ACP) to 1,3-butadiene through enzyme catalyzed steps involving the reduction of glutaconyl-CoA (or -ACP) to form the intermediate 5-hydroxypent-3-enoate. The disclosure provides alternative engineered pathway involving either decarboxylation of 5-hydroxypent-3-enoate directly to 1,3-butadiene, or phosphorylation of 5-hydroxypent-3-enoate followed by a phosphate elimination step catalyzed by a decarboxylase to produce 1,3-butadiene.

### 1. TECHNICAL FIELD

The present disclosure relates to recombinant host cells comprising one or more recombinant polynucleotides encoding enzymes in select pathways that provide the ability to use the cells to produce 1,3-butadiene, and the methods of manufacture of the cells, and methods of use of the cells for the production of 1,3-butadiene.

### 2. REFERENCE TO SEQUENCE LISTING

The official copy of the Sequence Listing is submitted concurrently with the specification as an ASCII formatted text file via EFS-Web, with a file name of “CX5-112USP1.txt”, a creation date of Mar. 1, 2012, and a size of 17,642 bytes. The Sequence Listing filed via EFS-Web is part of the specification and is incorporated in its entirety by reference herein.

### 3. BACKGROUND

1,3-butadiene (also referred to herein as “butadiene”) is a feedstock chemical used in the production synthetic rubbers, polymer resins, and other industrially important chemicals such as hexamethylenediamine, and adipidonitrile. Currently, nearly all of the 25 billion pounds of 1,3-butadiene produced annually is made by steam-cracking of non-renewable petroleum feedstock chemicals. Accordingly, there is a need for alternative processes that could produce 1,3-butadiene from renewable non-petroleum feedstock chemicals such as sugars (e.g., molasses, sugar cane juice), and particularly, from sugar compositions obtained from non-food cellulosic biomass sources (e.g., sugar cane bagasse, corn stover, wheat straw).

US2011/0300597A1 discloses non-naturally occurring microbial organisms containing butadiene pathways comprising at least one exogenous nucleic acid encoding a butadiene pathway enzyme expressed in a sufficient amount to produce butadiene. US2011/0300597A1 proposes, among other pathways, an engineered butadiene pathway that proposes starting with glutaconyl-CoA and using a glutaconyl-CoA decarboxylase to form crotonyl-CoA (see e.g. at FIG. 2, Step L, and paragraph [0159]). US2011/0300597A1 further proposes that the crotonyl-CoA is then subsequently reduced to crotonol in two steps, which then is activated as the pyrophosphate (2-butenyl-4-diphosphate) in two steps with two different kinase enzymes. The 2-butenyl-4-diphosphate is converted to butadiene in a final step using isoprene synthase (see e.g. FIG. 2, Steps F, G, and H, and paragraphs [0134]-[0140]).

US2012/0021478A1 discloses non-naturally occurring microbial organisms containing butadiene pathways comprising at least one exogenous nucleic acid encoding a butadiene pathway enzyme expressed in a sufficient amount to produce butadiene. US2012/0021478A1 proposes, among other pathways, an engineered butadiene pathway in which a 3,5-dihydroxypentanoate and/or a 5-hydroxypent-2-enoate intermediate is formed. This intermediate is then either decarboxylated by a supposed 3-hydroxyacid decarboxylase to form 3-butene-1-ol, or dehydrated to form 2,4-pentadienoate. The 3-butene-1-ol is subsequently dehydrated by a supposed 3-butene-1-ol dehydrogenase or a chemical catalyst to provide butadiene, and the 2,4-pentadienoate is further decarboxylated by a supposed 2,4-pentadiene decarboxylase to yield butadiene (see e.g., FIGS. 17 and 21, and paragraphs [0521]-[0523] and [0529]-[0531]).

### 4. SUMMARY

The present disclosure fulfills a need in the art by providing recombinant host cells that comprise an engineered pathway of enzymes as depicted in FIG. 1 or FIG. 2. The engineered pathway of enzymes are capable of catalyzing the series of conversions of substrate to product as depicted in FIG. 1 or FIG. 2, and the enzyme are encoded by one or more recombinant polynucleotides.

In some embodiments, the present disclosure provides a recombinant host cell capable of producing 1,3-butadiene, the host cell comprising: (a) a recombinant polynucleotide encoding an enzyme capable of converting crotonyl-CoA (or -ACP) to glutaconyl-CoA (or -ACP); and (b) a recombinant polynucleotide encoding an enzyme capable of converting glutaconyl-CoA (or -ACP) to 5-hydroxypent-3-enoate. In some embodiments, the recombinant host cell further comprises: (c) a recombinant polynucleotide encoding an enzyme capable of converting 5-hydroxypent-3-enoate to 1,3-butadiene. In other embodiments, the recombinant host cell further comprises: (c) one or more recombinant polynucleotides encoding an enzyme capable of converting 5-hydroxypent-3-enoate to 5-(phosphonatooxy)pent-3-enoate; and (d) an enzyme capable of converting 5-(phosphonatooxy)pent-3-enoate to 1,3-butadiene.

In some embodiments of the recombinant host cell, the recombinant polynucleotide encoding the FAR enzyme comprises one or more nucleotide sequence differences relative to the corresponding naturally occurring polynucleotide, which result in an improved property selected from: (a) increased activity of the FAR enzyme in the conversion of glutaconyl-CoA (or -ACP) to 5-hydroxypent-3-enoate: (b) increased expression of the FAR enzyme: (c) increased host cell tolerance of crotonyl-CoA (or -ACP), glutaconyl-CoA (or -ACP), 5-hydroxypent-3-enoate, 5-(phosphonatooxy)pent-3-enoate, or 1,3-butadiene; or (d) altered host cell concentration of crotonyl-CoA (or -ACP), glutaconyl-CoA (or -ACP), 5-hydroxypent-3-enoate, 5-(phosphonatooxy)pent-3-enoate, or 1,3-butadiene.

In further embodiments of the recombinant host cell, the recombinant polynucleotide encoding an FAR enzyme comprises a polynucleotide sequence that has at least 80%, at least 85%, at least 90%, at least 95%, at least 96%, at least 97%, at least 98%, at least 99%, or more, identity to a sequence encoding any one of SEQ ID NO: 1, 2, 3, and 4, or which hybridizes under stringent conditions to a polynucleotide sequence encoding any one of SEQ ID NO: 1, 2, 3, and 4. In some embodiments, the FAR enzyme comprises an amino acid sequence having at least 80%, at least 85%, at least 90%, at least 95%, at least 96%, at least 97%, at least 98%, at least 99%, or more, identity to an amino acid sequence of any one of SEQ ID NO: 1, 2, 3, and 4.

In some embodiments of the recombinant host cell, the recombinant polynucleotide encoding (i) the enzyme capable of converting crotonyl-CoA (or -ACP) to glutaconyl-CoA (or -ACP), (ii) the enzyme capable of converting 5-hydroxypent-3-enoate to 1,3-butadiene, (iii) converting 5-hydroxypent-3-enoate to 5-(phosphonatooxy)pent-3-enoate, and/or (iv) the enzyme capable of converting 5-(phosphonatooxy)pent-3-enoate to 1,3-butadiene, comprises one or more nucleotide sequence differences relative to the corresponding naturally occurring polynucleotide, which result in an improved property selected from: (a) increased activity of the enzyme in the conversion of its respective substrate to product: (b) increased expression of the enzyme: (c) increased host cell tolerance of crotonyl-CoA (or -ACP), glutaconyl-CoA (or -ACP), 5-hydroxypent-3-enoate, 5-(phosphonatooxy)pent-3-enoate, or 1,3-butadiene; or (d) altered host cell concentration of crotonyl-CoA (or -ACP), glutaconyl-CoA (or -ACP), 5-hydroxypent-3-enoate, 5-(phosphonatooxy)pent-3-enoate, or 1,3-butadiene.

In some embodiments of the recombinant host cell, one or more of (i) the enzyme capable of converting crotonyl-CoA (or -ACP) to glutaconyl-CoA (or -ACP), (ii) the enzyme capable of converting glutaconyl-CoA (or -ACP) to 5-hydroxypent-3-enoate, (iii) the enzyme capable of converting 5-hydroxypent-3-enoate to 1,3-butadiene, (iv) converting 5-hydroxypent-3-enoate to 5-(phosphonatooxy)pent-3-enoate, and/or (v) the enzyme capable of converting 5-(phosphonatooxy)pent-3-enoate to 1,3-butadiene, is a naturally occurring enzyme listed in any one of Tables 2, 3, 5, 6, 8, 10, or 11 disclosed herein, or an engineered enzyme derived from a naturally occurring enzyme listed in any one of Tables 2, 3, 5, 6, 8, 10, or 11 disclosed herein.

In some embodiments of the recombinant host cell, the host cell is capable of producing 1,3-butadiene by fermentation of a carbon source, wherein the carbon source is a fermentable sugar. In some embodiments, the fermentable sugar is glucose. In some embodiments, the fermentable is obtained from a cellulosic biomass, such as sugar cane bagasse, corn stover, or wheat straw.

In some embodiments of the recombinant host cell, the host cell is from a strain of microorganism derived from any one of: Escherichia coli, Bacillus, Saccharomyces, Streptomyces and Yarrowia. In some embodiments, the host cell is from a microorganism selected from E. coli, S. cerevisiae. and Y. lipolytica.

The present disclosure also provides methods of manufacturing the recombinant host cells of the disclosure (i.e., recombinant host cells comprising an engineered pathway of FIG. 1 or FIG. 2). In some embodiments, the method of manufacturing the recombinant host cell comprises transforming a suitable host cell with one or more nucleic acid constructs encoding: (a) a recombinant polynucleotide encoding an enzyme capable of converting crotonyl-CoA (or -ACP) to glutaconyl-CoA (or -ACP); (b) a recombinant polynucleotide encoding an enzyme capable of converting glutaconyl-CoA (or -ACP) to 5-hydroxypent-3-enoate; and (c) one or more recombinant polynucleotides encoding an enzyme capable of converting 5-hydroxypent-3-enoate to 5-(phosphonatooxy)pent-3-enoate. In other embodiments, the method of manufacturing the recombinant host cell comprises transforming a suitable host cell with one or more nucleic acid constructs encoding: (a) a recombinant polynucleotide encoding an enzyme capable of converting crotonyl-CoA (or -ACP) to glutaconyl-CoA (or -ACP); (b) a recombinant polynucleotide encoding an enzyme capable of converting glutaconyl-CoA (or -ACP) to 5-hydroxypent-3-enoate; (c) one or more recombinant polynucleotides encoding an enzyme capable of converting 5-hydroxypent-3-enoate to 5-(phosphonatooxy)pent-3-enoate; and (d) an enzyme capable of converting 5-(phosphonatooxy)pent-3-enoate to 1,3-butadiene.

The present disclosure also provides methods of using the recombinant host cells disclosed herein in processes for making 1,3-butadiene. In some embodiments, the disclosure provides a method of producing 1,3-butadiene comprising contacting a recombinant host cell of the disclosure (i.e. a recombinant host cell comprising an engineered pathway of FIG. 1 or FIG. 2) with a medium comprising a fermentable carbon source under suitable conditions for generating 1,3-butadiene. In some embodiments, the method further comprises a step of recovering the 1,3-butadiene produced by the recombinant host cell. In some embodiments of the method, the carbon source comprises a fermentable sugar, optionally wherein the fermentable sugar is selected from glucose, and a fermentable sugar obtained from biomass, such as sugar cane bagasse, corn stover, or wheat straw.

### 5. BRIEF DESCRIPTION OF THE DRAWINGS

FIG. 1 depicts schematically a pathway of enzymes capable of converting crotonyl-CoA (or -ACP) to 1,3-butadiene. The pathway includes three catalytic steps A, B, and E. Step A is the conversion of crotonyl-CoA (or -ACP) to glutaconyl-CoA (or -ACP) by a carboxylase enzyme (EC 6.4.1.x); Step B is the conversion of glutaconyl-CoA (or -ACP) to 5-hydroxypent-3-enoate by a single fatty acyl reductase (FAR) enzyme (EC 1.1.1*); and Step E is the conversion of 5-hydroxypent-3-enoate to 1,3-butadiene by a dehydratase enzyme (EC 4.2.1.x). Steps C and D depict an alternative pathway that utilizes a pair of enzymes to convert glutaconyl-CoA (or -ACP) to 5-hydroxypent-3-enoate through formation of the aldehyde intermediate, 5-oxopent-3-enoate. Enzymes that convert, or that can be engineered to convert, the depicted substrate to product at each of the steps in the pathways are described in further detail herein.

FIG. 2 depicts schematically a pathway of enzymes capable of converting crotonyl-CoA (or -ACP) to 1,3-butadiene. The pathway includes four catalytic steps A, B, E, and F. Step A is the conversion of crotonyl-CoA (or -ACP) to glutaconyl-CoA (or -ACP) by a carboxylase enzyme (EC 6.4.1.x); Step B is the conversion of glutaconyl-CoA (or -ACP) to 5-hydroxypent-3-enoate by a FAR enzyme (EC 1.1.1*): Step E is the conversion of 5-hydroxypent-3-enoate to 5-(phosphonatooxy)pent-3-enoate by a kinase enzyme (EC 2.7.1.x); and Step F is the conversion of the phosphate, 5-(phosphonatooxy)pent-3-enoate directly to 1,3-butadiene by a decarboxylase enzyme (EC 4.1.1.x). Steps C and D depict an alternative pathway that utilizes a pair of enzymes to convert glutaconyl-CoA (or -ACP) to 5-hydroxypent-3-enoate through formation of the aldehyde intermediate, 5-oxopent-3-enoate. Enzymes that convert, or that can be engineered to convert, the depicted substrate to product at each of the steps in the pathways are described in further detail herein.

### 6. DETAILED DESCRIPTION

The present disclosure addresses the need in the art for biological compositions and associated methods to produce 1,3-butadiene from cheap, renewable carbon sources, such as fermentable sugars obtained from plant biomass.

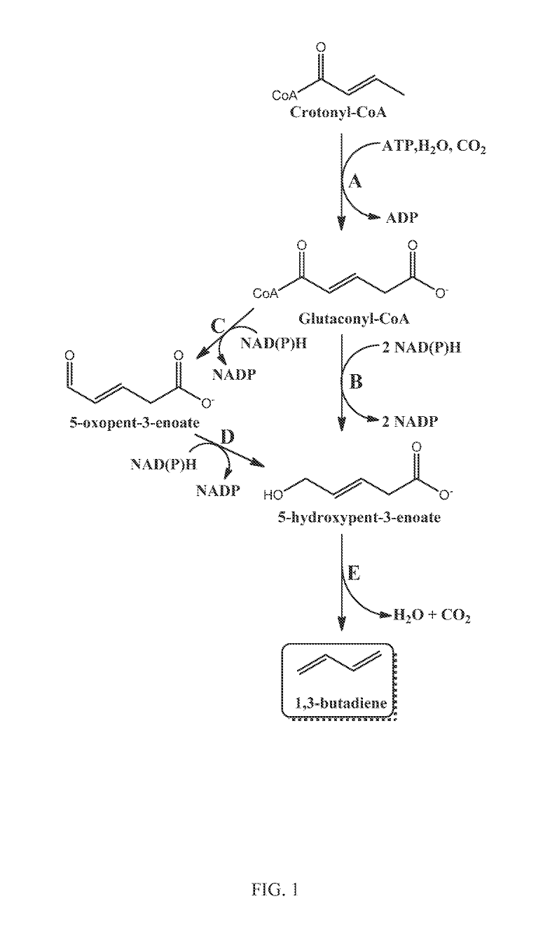

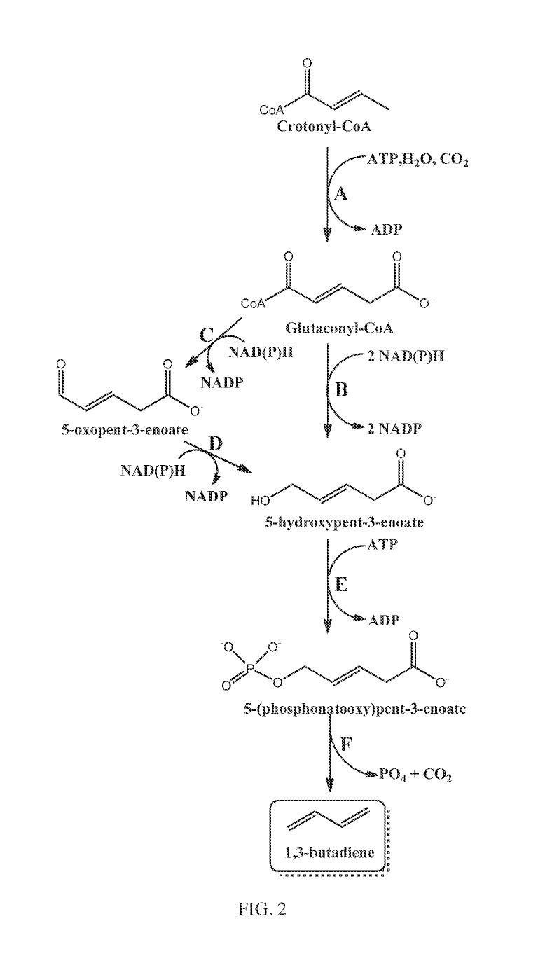

The present disclosure provides recombinant host cells that are capable of producing 1,3-butadiene via an engineered pathway through a 5-hydroxypent-3-enoate intermediate, and associated compositions, processes, techniques, and methods of manufacture, that can provide for large scale production of 1,3-butadiene. The recombinant host cells of the disclosure comprise one or more recombinant polynucleotides that encode one or more enzymes in select pathways of enzymes, which are depicted schematically in FIG. 1 and FIG. 2. The functioning of these engineered pathways of enzymes provide the recombinant host cells with the ability to produce 1,3-butadiene.

In particular embodiments, the recombinant host cells comprise a recombinant polynucleotide encoding a fatty acyl reductase (FAR) enzyme which as a single enzyme is capable of converting acyl-CoA (or -ACP) compound, glutaconyl-CoA (or -ACP) to the alcohol compound, 5-hydroxypent-3-enoate. In some embodiments, the FAR enzyme is an engineered enzyme derived from a fatty acyl reductase gene found in a species of Marinobacter or Oceanobacter, and in particular embodiments the gene found in Marinobacter algicola strain DG893 or Marinobacter aquaeolei VT8.

In some embodiments of the disclosure, the recombinant host cells comprise a recombinant polynucleotide encoding a dehydratase enzyme that carries out the step of converting of 5-hydroxypent-3-enoate to 1,3-butadiene (as in FIG. 1, Step E). This engineered pathway is depicted in FIG. 1, and the enzymes are further described herein. The present disclosure contemplates that the activity, selectivity and stability of each of the enzymes involved can be improved and/or modified via standard directed evolution/enzyme engineering techniques.

In some embodiments of the disclosure, the recombinant host cells further comprise an engineered pathway of enzymes that carry out the further two steps of a kinase catalyzed conversion of 5-hydroxypent-3-enoate to the phosphate compound, 5-(phosphonatooxy)pent-3-enoate, and a decarboxylase catalyzed phosphate elimination of 5-(phosphonatooxy)pent-3-enoate to 1,3-butadiene (as in FIG. 2, Steps E and F), thereby providing an alternative biosynthetic route for the production 1,3-butadiene. This engineered pathway is depicted in FIG. 2, and the enzymes are further described herein. The present disclosure contemplates that the activity, selectivity and stability of each of the enzymes involved can be improved and/or modified via standard directed evolution/enzyme engineering techniques.

In some embodiments, the recombinant host cells comprise one or more recombinant polynucleotides encoding an engineered variant of an enzyme described herein and in the engineered pathways of FIGS. 1 and 2. These engineered variants of enzymes can have an improved property relative to the corresponding reference sequence from which they are derived, and be generated using standard techniques of enzyme engineering (e.g., gene shuffling, directed evolution).

The recombinant host cells, engineered pathways, and specific recombinant polynucleotides and encoded enzymes that make up the pathways and carry out the substrate-to-product conversions are described in greater detail below. Additionally, the following sections describe methods for using the recombinant host cells for the production of 1,3-butadiene from fermentable sugars.

### 6.1. DEFINITIONS

The technical and scientific terms used in the descriptions herein will have the meanings commonly understood by one of ordinary skill in the art, unless specifically defined otherwise. Accordingly, the following terms are intended to have the following meanings.

“Protein”, “polypeptide,” and “peptide” are used interchangeably herein to denote a polymer of at least two amino acids covalently linked by an amide bond, regardless of length or post-translational modification (e.g. glycosylation, phosphorylation, lipidation, myristilation, ubiquitination, etc.). Included within this definition are D- and L-amino acids, and mixtures of D- and L-amino acids.

“Enzyme” as used herein refers to a polypeptide or protein having capable of catalyzing the conversion of substrate molecule to a product molecule.

“Nucleic acid” or “polynucleotide” are used interchangeably herein to denote a polymer of at least two nucleic acid monomer units or bases (e.g. adenine, cytosine, guanine, thymine) covalently linked by a phosphodiester bond, regardless of length or base modification

“Naturally occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

“Recombinant” or “engineered” or “non-naturally occurring” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. Mol. Biol. 215: 403-410 and Altschul et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison Wis.), using default parameters provided.

“Reference sequence” refers to a defined sequence to which another sequence is compared. A reference sequence is not limited to wild-type sequences, and can include engineered or altered sequences. For example, a reference sequence can be a previously engineered or altered amino acid sequence. A reference sequence also may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered enzyme, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Different from” or “differs from” with respect to a designated reference sequence refers to difference of a given amino acid or polynucleotide sequence when aligned to the reference sequence. Generally, the differences can be determined when the two sequences are optimally aligned. Differences include insertions, deletions, or substitutions of amino acid residues in comparison to the reference sequence. Typically, the reference sequence is a naturally occurring sequence from which the sequence with the differences is derived. The present disclosure provides engineered pathways of enzymes, wherein the enzymes are encoded by one or more recombinant polynucleotides having one or more nucleotide sequence differences relative to a reference polynucleotide sequence, which is typically the corresponding naturally occurring polynucleotide from which the recombinant polynucleotide is derived. Further, the nucleotide differences may encode one or more amino acid residue differences in the enzymes, where the encoded amino acid differences, which can include either/or both conservative and non-conservative amino acid substitutions.

“Derived from” as used herein in the context of engineered enzymes, identities the originating enzyme, and/or the gene encoding such enzyme, upon which the engineering was based.

“Amino acid residue” or “amino acid” or “residue” as used herein refers to the specific monomer at a sequence position of a polypeptide.

“Amino acid difference” or “residue difference” refers to a change in the amino acid residue at a position of a polypeptide sequence relative to the amino acid residue at a corresponding position in a reference sequence.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid, e.g. alanine, valine, leucine, and isoleucine; an amino acid with hydroxyl side chain is substituted with another amino acid with a hydroxyl side chain. e.g., serine and threonine; an amino acids having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine and arginine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Non-conservative substitution” refers to substitution of an amino acid in a polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification of the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the polypeptide while retaining enzymatic activity and/or retaining the improved properties of an engineered enzyme. Deletions can be directed to the internal portions and/or terminal portions of the polypeptide. In various embodiments, the deletion can comprise a continuous segment or can be discontinuous.

“Insertion” refers to modification of the polypeptide by addition of one or more amino acids to the reference polypeptide. In some embodiments, the improved engineered enzymes comprise insertions of one or more amino acids relative to the corresponding naturally occurring polypeptide as well as insertions of one or more amino acids to other improved polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Fragment” as used herein refers to a polypeptide that has an amino-terminal and/or carboxy-terminal deletion, but where the remaining amino acid sequence is identical to the corresponding positions in the sequence. Fragments can typically have about 80%, 90%, 95%, 98%, and 99% of the full-length polypeptide, for example the FAR enzyme polypeptide of SEQ ID NO: 1. The amino acid sequences of the specific recombinant polypeptides included in the Sequence Listing of the present disclosure include an initiating methionine (M) residue (i.e., M represents residue position 1). The skilled artisan, however, understands that this initiating methionine residue can be removed by biological processing machinery, such as in a host cell or in vitro translation system, to generate a mature protein lacking the initiating methionine residue, but otherwise retaining the enzyme's properties. Consequently, the term “amino acid residue difference relative to SEQ ID NO: 1 at position n” as used herein may refer to position “n” or to the corresponding position (e.g. position (n−1) in a reference sequence that has been processed so as to lack the starting methionine.

“Improved property” as used herein refers to a functional characteristic of an enzyme or host cell that is improved relative to the same functional characteristic of a reference enzyme or reference host cell. Improved properties of the engineered enzymes or host cells comprising engineered pathways disclosed herein can include but are not limited to: increased thermostability, increased solvent stability, increased pH stability, altered pH activity profile, increased activity (including increased rate conversion of substrate to product, or increased percentage conversion in a period of time), increased and/or altered stereoselectivity, altered substrate specificity and/or preference, decreased substrate, product, and side-product inhibition, decreased inhibition by a component of a feedstock, decreased side-product or impurity production, altered cofactor preference, increased expression, increased secretion, as well as increased stability and/or activity in the presence of additional compounds reagents useful for the production of 1,3-butadiene.

“Stability in the presence of” as used in the context of improved enzyme properties disclosed herein refers to stability of the enzyme measured during or after exposure of the enzyme to certain compounds/reagents/ions in the same solution with the enzyme. It is intended to encompass challenge assays of stability where the enzyme is first exposed to the compounds/reagents/ions for some period of time then assayed in a solution under different conditions.

“Solution” as used herein refers to any medium, phase, or mixture of phases, in which the recombinant host cells and/or enzymes of the present disclosure is active. It is intended to include purely liquid phase solutions (e.g. aqueous, or aqueous mixtures with co-solvents, including emulsions and separated liquid phases), as well as slurries and other forms of solutions having mixed liquid-solid phases.

“Thermostability” refers to the functional characteristic of retaining activity (e.g., more than 60% to 80%) in the presence of, or after exposure to for a period of time (e.g. 0.5-72 hrs), elevated temperatures (e.g. 30-60° C.) compared to the activity of an untreated enzyme.

“Solvent stability” refers to the functional characteristic of retaining activity (e.g., more than 60% to 80%) in the presence of, or after exposure to for a period of time (e.g. 0.5-72 hrs), increased concentrations (e.g., 5-99%) of solvent compared to the activity of an untreated enzyme.

“pH stability” refers to the functional characteristic of retaining activity (e.g., more than 60% to 80%) in the presence of, or after exposure to for a period of time (e.g. 0.5-72 hrs), conditions of high or low pH (e.g., pH 2 to 12) compared to the activity of an untreated enzyme.

“Increased activity” or “increased enzymatic activity” refers to an improved property of an enzyme (e.g., FAR enzyme), which can be represented by an increase in specific activity (e.g., product produced/time/weight protein) or an increase in percent conversion of the substrate to the product (e.g., percent conversion of glutaconyl-CoA to 5-hydroxypent-3-enoate in a specified time period using a specified amount of a FAR enzyme) as compared to a reference enzyme under suitable reaction conditions. Any property relating to enzyme activity may be altered, including the classical enzyme properties of Kₘ, Vₘₐₓ or k_{cat}, changes of which can lead to increased enzymatic activity. Improvements in enzyme activity can be from about 1.1-times the enzymatic activity of the corresponding naturally occurring enzyme, to as much as 1.2-times, 1.5-times, 2-times, 3-times, 4-times, 5-times, 6-times, 7-times, or more than 8-times the enzymatic activity than the naturally occurring parent enzyme. It is understood by the skilled artisan that the activity of any enzyme is diffusion limited and hence, any improvements in the enzyme activity of the enzyme will have an upper limit related to the diffusion rate of the substrates acted on by the enzyme. Methods to determine enzyme activity can depend on the particular enzyme, substrate, and product, and are well-known in the art. Comparisons of enzyme activities are made, e.g., using a defined preparation of enzyme, a defined assay under a set of conditions, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Conversion” refers to the enzymatic conversion of the substrate to the corresponding product. “Percent conversion” refers to the percent of the substrate that is reduced to the product within a period of time under specified conditions. Thus, the “enzymatic activity” or “activity” of a enzyme can be expressed as “percent conversion” of the substrate to the product.

“Isolated” as used herein in the context of enzymes or compounds such as “isolated 5-hydroxypent-3-enoate” refers to a molecule which is substantially separated from other contaminants that naturally accompany it. The term embraces isolated compounds, such as isolated 5-hydroxypent-3-enoate, which have been made biosynthetically in a recombinant host cell and then are removed or purified from the cellular environment or expression system.

“Coding sequence” refers to that portion of a polynucleotide that encodes an amino acid sequence of a protein (e.g., a gene).

“Heterologous” polynucleotide refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. In some embodiments, the polynucleotides encoding the enzymes used in the engineered pathways of the present disclosure may be codon optimized for optimal production from the host organism selected for expression.

“Control sequence” is defined herein to include all components, which are necessary or advantageous for the expression of a polynucleotide and/or polypeptide of the present disclosure. Each control sequence may be native or foreign to the polynucleotide of interest. Such control sequences include, but are not limited to, a leader, polyadenylation sequence, propeptide sequence, promoter, signal peptide sequence, and transcription terminator.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Expression” includes any step involved in the production of a polypeptide (e.g., encoded enzyme) including, but not limited to, transcription, post-transcriptional modification, translation, post-translational modification, and secretion.

“Transform” or “transformation.” as used in reference to a host cell, means a host cell has a non-native nucleic acid sequence integrated into its genome or as an episome (e.g., plasmid) that is maintained through multiple generations of the host cell.

“Culturing” refers to growing a population of host cells under suitable conditions in a liquid or solid medium. In particular embodiments, culturing refers to the fermentative bioconversion of a carbon source (e.g., sugar) to an end product (e.g., butadiene).

“Recoverable” as used in reference to producing a composition (e.g., 1,3-butadiene) by a method of the present invention, refers to the amount of composition which can be isolated from the reaction mixture yielding the composition according to methods known in the art.

“Enzyme class” as used herein refers to the numerical classification scheme for enzymes based on the reaction catalyzed by the enzyme. The enzyme class is designated by the Enzyme Commission (“EC”) number. The EC number classification scheme is well-known in the art and published by International Union of Biochemistry and Molecular Biology (IUBMB) (see at e.g., www.chem.qmul.ac.uk/iubmb/enzyme).

“Pathway of enzymes” or “enzyme pathway” refers to a group of enzymes expressed in a host cell that catalyze a series of conversions of substrate to product that are linked together, e.g., the product of the first enzyme is the substrate for the second enzyme, and the product of the second enzyme is the substrate of the third enzyme, and so on. As used herein, the term enzyme pathway may refer to a naturally occurring or an engineered pathway. Further, as used herein, an enzyme pathway may be part of a larger pathway in a cell (i.e., a sub-pathway).

“Host cell” as used herein refers to a living cell or microorganism that is capable of reproducing its genetic material and along with it recombinant genetic material that has been introduced into it e.g., via heterologous transformation.

“Recombinant host cell” as used herein refers to a host cell that has been transformed with recombinant genetic material—e.g., one or more recombinant polynucleotides.

“Sugar” as used herein refers to carbohydrate compounds and compositions made up of monosaccharides, disaccharides, trisaccharides, oligosaccharides, and polysaccharides, e.g., fructose, galactose, glucose, ribose, xylose, sucrose, lactose, maltose, maltotriose, starch, cellulose.

“Fermentable sugar” as used herein refers to sugar compounds and compositions that can be metabolized by a recombinant host cell. Exemplary fermentable sugars include sugars from sugar cane, starch from feedstock such as corn, from lignocellulosic feedstocks where the cellulose part of a plant is broken down to sugars (e.g. in a saccharification process) glucose and xylose.

“1,3-Butadiene” or “butadiene” as used herein refers to the diene compound of molecular formula C₄H₆ having CAS number 106-99-0. IUPAC name: buta-1,3-diene.

“CoA” as used herein refers to coenzyme A, the naturally occurring thiol compound having CAS number 85-61-0.

“ACP” as used herein refers to the acyl carrier protein, the naturally occurring polypeptide that comprises 4′-phosphopantethiene moiety which can forms a thioester linkage with the growing fatty acid chain during the biosynthesis of fatty acids.

“Crotonyl-CoA” or “crotonoyl-CoA” as used herein refers to the thioester compound of crotonyl (either the trans- or the cis-isomer or a mixture thereof) and coenzyme A which has the CAS number 992-67-6. IUPAC name: S-[2-[3-[[4-[[[5-(6-aminopurin-9-yl)-4-hydroxy-3-phosphonooxyoxolan-2-yl]methoxy-hydrxyphosphoryl]oxy-hydroxyphosphoryl]oxy-2-hydroxy-3,3-dimethylbutanoyl]amino]propanoylamino]ethyl]but-2-enethioate.

“Crotonyl-ACP” or “crotonoyl-ACP” as used herein refers to the compound of a crotonyl moiety (either the trans- or the cis-isomer or a mixture thereof) attached through a thioester linkage to the acyl-carrier protein.

“Glutaconyl-CoA” as used herein refers to the thioester compound of glutaconyl (either the trans- or the cis-isomer or a mixture of trans- and cis-) and coenzyme A which has the CAS number 6712-05-6. IUPAC name: 5-[2-[3-[[4-[[[5-(6-aminopurin-9-yl)-4-hydroxy-3-phosphonooxyoxolan-2-yl]methoxy-hydroxyphosphoryl]oxy-hydroxyphosphoryl]oxy-2-hydroxy-3,3-dimethylbutanoyl]amino]propanoylamino]ethylsulfanyl]-5-oxopent-3-enoic acid.

“5-Hydroxypent-3-enoate” as used herein refers to the allylic alcohol compound having the structure labeled “5-hydroxypent-3-enoate” in FIGS. 1 and 2, and includes either the trans- or the cis-isomer or a mixture thereof.

“5-Oxopent-3-enoate” as used herein refers to the aldehyde compound having the structure labeled “5-oxopent-3-enoate” in FIGS. 1 and 2, and includes either the trans- or the cis-isomer or a mixture thereof.

5-(Phosphonatooxy)pent-3-enoate” as used herein refers to the phosphate compound having the structure labeled “5-(phosphonatooxy)pent-3-enoate” in FIG. 2, and includes either the trans- or the cis-isomer or a mixture thereof.

“FAR enzyme” or “fatty acyl reductase” refers to an enzyme that catalyzes reduction of a fatty acyl-CoA, a fatty acyl-ACP, or other fatty acyl thioester substrate directly to its corresponding fatty alcohol with the hydride equivalents provided by the oxidation of NAD(P)H to NAD(P)⁺. (EC 1.1.1*) The enzymatic reaction catalyzed by a FAR enzyme on fatty acyl-CoA can be represented by:

fatty acyl-CoA+2NAD(P)H→fatty alcohol+2NAD(P)⁺

In contrast to the FAR enzyme, where a single enzyme catalyzes this reduction to the fatty alcohol more typically the enzymatic reduction of fatty acyl-CoA molecules to fatty alcohols is catalyzed two distinct reductase enzymes: (1) an “acyl-CoA reductase” which reduces the acyl-CoA substrate to its corresponding fatty aldehyde (e.g. enzyme of class EC 1.2.1.50); and (2) an “fatty aldehyde reductase” (e.g. an oxidoreductase) reduces the fatty aldehyde to the fatty alcohol (e.g., an enzyme of class EC 1.1.1.1). Such a two-enzyme reduction can be represented by:

fatty acyl-CoA+NAD(P)H→fatty aldehyde+NAD(P)⁺

fatty aldehyde+NAD(P)H→fatty alcohol+NAD(P)⁺

### 6.2. ENGINEERED PATHWAYS OF ENZYMES FOR BIOSYNTHETIC PRODUCTION OF 1,3-BUTADIENE

The present disclosure provides recombinant host cells comprising engineered pathways of enzymes that are useful for the production of 1,3-butadiene. Generally, the engineered pathways introduced into the host cells by transforming the host cells with one or more recombinant polynucleotides encoding one or more of the enzymes in the pathway. The recombinant host cells thereby produced are capable of expressing the encoded enzyme(s) such that the substrate-to-product conversions of the engineered pathway are carried out biosynthetically and host cell produces the desired product compound, 1,3-butadiene. The relevant portions of the engineered pathways are illustrated schematically in FIG. 1 and FIG. 2.

In some embodiments, the recombinant host cells comprising engineered pathways of enzymes are capable of producing the compound 1,3-butadiene from the metabolic compound, crotonyl-CoA (or -ACP), which is naturally occurring in the host cell. The engineered pathway of enzymes form 1,3-butadiene via the intermediate compounds glutaconyl-CoA (or -ACP) and 5-hydroxypent-3-enoate. In such embodiments, the recombinant host cell comprises a recombinant polynucleotide encoding an enzyme capable of converting crotonyl-CoA (or -ACP) to glutaconyl-CoA (see Step A of FIG. 1 or 2), and one or more enzymes capable of converting glutaconyl-CoA (or -ACP) to 5-hydroxypent-3-enoate (see Step B of FIG. 1 or 2).

In some embodiments, the recombinant polynucleotide encodes a single FAR enzyme capable of converting glutaconyl-CoA (or -ACP) to 5-hydroxypent-3-enoate (as in Step B of FIG. 1 or 2). In some embodiments, the FAR enzyme is an engineered enzyme derived from a fatty acyl reductase gene found in a species of Marinobacter or Oceanobacter, and in particular embodiments the gene found in Marinobacter algicola strain DG893 or Marinobacter aquaeolei VT8. In other embodiments, the recombinant host cell comprises a recombinant polynucleotide encoding a pair of enzymes capable of converting glutaconyl-CoA (or -ACP) to 5-hydroxypent-3-enoate through formation of the aldehyde intermediate, 5-oxopent-3-enoate. In another embodiment, the recombinant host cell comprises one or more recombinant polynucleotide encoding a pair of enzymes capable of converting glutaconyl-CoA (or -ACP) to 5-hydroxypent-3-enoate through formation of the aldehyde intermediate, 5-oxopent-3-enoate (as in Steps C and D of FIG. 1 or 2).

In some embodiments, the recombinant host cells comprising engineered pathways of enzymes are capable of producing 1,3-butadiene directly from the intermediate compound 5-hydroxypent-3-enoate. In such embodiments, the recombinant host cells comprise: a recombinant polynucleotide encoding a dehydratase enzyme capable of converting of 5-hydroxypent-3-enoate to 1,3-butadiene (as in FIG. 1, Step E).

In some embodiments, the recombinant host cells comprising engineered pathways of enzymes are capable of producing 1,3-butadiene in two enzyme catalyzed steps from the intermediate compound 5-hydroxypent-3-enoate. In such embodiments, the recombinant host cells comprise: (i) a recombinant polynucleotide encoding a kinase enzyme capable of converting 5-hydroxypent-3-enoate to 5-(phosphonatooxy)pent-3-enoate (see FIG. 2, Step E): and (ii) a recombinant polynucleotide encoding an decarboxylase enzyme capable of catalyzing the phosphate elimination of 5-(phosphonatooxy)pent-3-enoate to form 1,3-butadiene (see FIG. 2, Step F).

In some embodiments, the enzyme capable of converting 5-hydroxypent-3-enoate to 5-(phosphonatooxy)pent-3-enoate is an engineered alcohol kinase enzyme. In some embodiments, the enzyme capable of capable of converting 5-(phosphonatooxy)pent-3-enoate to 1,3-butadiene is an engineered mevalonate pyrophosphate decarboxylase enzyme.

The present disclosure contemplates that any of the exemplary enzymes disclosed herein may be engineered using methods known in the art (e.g. random PCR, gene shuffling, directed evolution, etc.) to provide variant engineered enzymes having improved properties. Specific improved properties of engineered enzymes useful for the recombinant host cells of the present disclosure can include altered (i.e., increased or decreased) enzyme activity or enzyme expression. For example, decreased enzyme activity or expression may be desirable in many situations, particularly to prevent the detrimental build-up in concentration of product which can be a substrate for another slower downstream enzyme in the pathway.

The engineered enzymes of the present disclosure can be obtained by subjecting the polynucleotide encoding the naturally occurring enzyme (or one or more homologous naturally occurring enzymes) to mutagenesis and/or directed evolution methods. Exemplary techniques for engineering enzymes of the present disclosure can include directed evolution techniques such as mutagenesis and/or DNA shuffling as described in Stemmer, 1994, Proc Natl Acad Sci USA 91:10747-10751; WO 95/22625: WO 97/0078: WO 97/35966; WO 98/27230; WO 00/42651; WO 01/75767 and U.S. Pat. No. 6,537,746. Other directed evolution procedures that can be used include, among others, staggered extension process (StEP), in vitro recombination (Zhao et al., 1998, Nat. Biotechnol. 16:258-261), mutagenic PCR (Caldwell et al., 1994, PCR Methods Appl. 3:S136-S140), and cassette mutagenesis (Black et al., 1996, Proc Natl Acad Sci USA 93:3525-3529). Mutagenesis and directed evolution techniques useful for the purposes herein are also described in e.g., Ling, et al., 1997, Anal. Biochem. 254(2): 157-78; Dale et al., 1996, “Oligonucleotide-directed random mutagenesis using the phosphorothioate method,” in Methods Mol. Biol. 57:369-74; Smith, 1985, Ann. Rev. Genet. 19:423-462; Botstein et al., 1985, Science 229:1193-1201; Carter, 1986, Biochem. J. 237:1-7; Kramer et al., 1984. Cell, 38:879-887; Wells et al., 1985, Gene 34:315-323; Minshull et al., 1999, Curr Opin Chem Biol 3:284-290; Christians et al., 1999, Nature Biotech 17:259-264; Crameri et al. 1998. Nature 391:288-291; Crameri et al., 1997, Nature Biotech 15:436-438; Zhang et al., 1997, Proc Natl Acad Sci USA 94:45-4-4509; Crameri et al. 1996. Nature Biotech 14:315-319; Stemmer, 1994, Nature 370:389-391; Stemmer, 1994, Proc Natl Acad Sci USA 91:10747-10751; PCT Publ. Nos. WO 95/22625, WO 97/0078. WO 97/35966, WO 98/27230. WO 00/42651, and WO 01/75767: and U.S. Pat. No. 6,537,746. All publications and patent are hereby incorporated by reference herein.

In some embodiments, it is contemplated that the enzymes disclosed herein are encoded by recombinant polynucleotides having sequences that have been codon optimized for expression in the recombinant host cell. In some embodiments, it is contemplated that the enzymes disclosed herein are encoded by recombinant polynucleotides having sequences that also include control sequences that can increase expression and/or secretion of the enzymes. The control sequences may be ones associated with the enzyme gene in its host organism or associated with the host cell. In some embodiments, it is contemplated that the recombinant polynucleotides that can further comprise a sequence encoding a signal peptide. In such embodiments, the signal peptide may be one that is associated with the enzyme in its naturally occurring organism. In other embodiments, the signal peptide can be one that is associated with a gene found in the recombinant host cell, thereby providing for the improved expression of the enzyme in the host cell.

Exemplary enzymes that can be used in the various substrate-to-product conversion steps of the engineered pathways of the present disclosure are described in greater detail below and in the Examples.

Pathway of FIG. 1 and FIG. 2, Step A

Crotonyl-CoA (or -ACP) is a naturally occurring metabolic intermediate formed in host cells via the fermentation of butyric acid and/or the metabolism of lysine or tryptophan. Crotonyl-CoA (or -ACP) can be carboxylated (i.e., addition of CO₂ or HCO₃⁻) to produce glutaconyl-CoA (or -ACP) by a carboxylase enzyme known for catalyzing the addition of CO₂ or HCO₃ to an acceptor molecule, such as an enzyme in a class EC 6.4.1 shown in Table 1.

TABLE 1

| EC | |
|---|---|
| Number | Enzyme Name |
| 6.4.1.1 | Pyruvate carboxylase |
| 6.4.1.2 | Acetyl-CoA carboxylase |
| 6.4.1.3 | Propionyl-CoA carboxylase |
| 6.4.1.4 | Methylcrotonyl-CoA carboxylase |
| 6.4.1.5 | Geranoyl-CoA carboxylase |
| 6.4.1.6 | Acetone carboxylase |
| 6.4.1.7 | 2-oxoglutarate carboxylase |
| 6.4.1.8 | Acetophenone carboxylase |

Exemplary carboxylase enzymes in the classes EC 6.4.1.4 and EC 6.4.1.5 that could be used in preparing an engineered pathway of FIG. 1 or 2, Step A of the present disclosure are shown in Table 2.

TABLE 2

| | | | | GI |
|---|---|---|---|---|
| Gene | Organism | UniProt id | GenBank id | Number |
| Mccc1 | Mus musculus | Q99MR8 | AF313338.1 | 12276064 |
| Mccc2 | Mus musculus | Q3ULD5 | AK132265.1 | 74205533 |
| MCCA | Glycine max | Q42777 | AAA53141.1 | 497234 |
| MCCB | Arabidopsis thaliana | Q9LDD8 | AF059511.1 | 7021224 |
| atuF | Pseudomonas | Q9HZV6 | AAG06279.1 | 9948982 |
| | aeruginosa | | | |
| atuC | Pseudomonas | Q9HZV6 | AAG06276.1 | 9948979 |
| | aeruginosa | | | |

In some embodiments of the present disclosure, an enzyme of Table 2 naturally occurs in the host cell used to prepare the recombinant host cell capable of producing 1,3-butadiene. In such an embodiment, no further modification of the host cell is needed to provide expression of an enzyme capable of the conversion of substrate to product of Step A of FIG. 1 or FIG. 2. In certain embodiments, a naturally occurring gene, or a natural homolog of such a gene, encoding an enzyme of Table 2 can be used to heterologously transform a host cell which lacks such a gene, and/or has such a gene but the native gene expresses either too little or too much of the desired enzyme. Accordingly, heterologous transformation with a gene of Table 2 can provide a recombinant host cell with an improved property (e.g., altered expression of a gene, altered concentration of a substrate and/or product due to use of a non-native gene in the pathway). In certain embodiments, an engineered version of a gene of Table 2 can be used to transform a host cell to provide an enzyme capable of the conversion of substrate to product of Step A of FIG. 1 or FIG. 2 having an improved property (e.g., increased conversion of the specific substrate of Step A).

Pathway of FIG. 1 and FIG. 2, Step B—Single-Enzyme Reduction of Glutaconyl-CoA (or -ACP) to 5-Hydroxypent-3-Enoate

In some embodiments, the conversion of glutaconyl-CoA (or -ACP) to 5-hydroxypent-3-enoate at Step B of the pathways of FIG. 1 and FIG. 2, is carried out by a single fatty acyl reductase (“FAR”) enzyme or a functional fragment thereof. The conversion of a fatty acyl-CoA (or -ACP) to its corresponding fatty alcohol requires four reducing equivalents (two hydrides) and thus, typically is carried out by two different NADPH dependent enzymes, e.g. an acyl-CoA reductase and a fatty aldehyde reductase. In contrast, a single FAR enzyme can catalyze the direct reduction of a fatty acyl-CoA (or -ACP) directly to its corresponding fatty alcohol, with the aldehyde forming only transiently in the active site, if at all, and not being released into solution (see e.g., Hofvander et al., “A prokaryotic acyl CoA reductase performing reduction of fatty acyl-CoA to fatty alcohol.” FEBS Letters 585: 3538-3543 (2011), which is hereby incorporated by reference herein).

A number of FAR enzymes obtained from marine bacteria, and engineered enzyme variants thereof, which are useful in preparing the recombinant host cells and methods of the present disclosure are disclosed in International patent publication WO2012/006114, which is hereby incorporated by reference herein. Further detailed description of useful FAR enzymes is provided below.

In certain embodiments, the FAR enzyme and/or functional fragment can be derived or obtained from a γ proteobacterium of the order Alteromonadales. In some embodiments, the FAR enzyme and/or functional fragment can be derived from or obtained from the Alteromonadales family Alteromonadaceae. In certain embodiments, the FAR enzyme and/or functional fragment can be derived from or obtained from an Alteromonadaceae genus such as but not limited to the Alteromonadaceae genus Marinobacter. In certain specific embodiments, the FAR enzyme and/or functional fragment can be derived from the Marinobacter species algicola. In a particular embodiment, the FAR enzyme and/or functional fragment can be derived from or obtained from the M. algicola species strain DG893. In some specific embodiments, the FAR enzyme for use in the methods disclosed herein is from the marine bacterium Marinobacter algicola DG893 (SEQ ID NO: 1) (“FAR_Maa”).

In some embodiments, the FAR enzyme and/or functional fragment is derived or obtained from a species of Marinobacter including, but not limited to, a species selected from M. algicola, M. alkaliphilus, M. aquaeolei, M. arcticus, M. bryozoorum, M. daepoensis, M. excellens, M. flavimaris, M. guadonensis, M. hydrocarbonoclasticus, M. koreenis, M. lipolyticus, M. litoralis, M. lutaoensis, M. maritimus, M. sediminum, M. squalenivirans and M. vinifirmus and equivalent and synonymous species thereof.

In one specific embodiment, the FAR enzyme is derived or obtained from M. algicola strain DG893 and has an amino acid sequence that is at least 70% identical, at least 75%, at least 80% identical, at least 85% identical, at least 90% identical, at least 93% identical at least 95% identical, at least 97% identical and/or at least 98% identical to SEQ ID NO: 1 or a functional fragment thereof. In another specific embodiment, the isolated FAR enzyme has an amino acid sequence that is identical to SEQ ID NO: 1.

In one specific embodiment, the FAR enzyme is derived or obtained from Marinobacter aquaeolei (e.g., M. aquaeolei VT8) and has an amino acid sequence that is at least at least 70% identical, at least 75%, at least 80% identical, at least 85% identical, at least 90% identical, at least 93% identical, at least 95% identical, at least 97% identical and/or at least 98% identical to SEQ ID NO: Y or a functional fragment thereof. In another specific embodiment, the isolated FAR enzyme has an amino acid sequence that is identical to SEQ ID NO: 2.

In various embodiments, the isolated FAR enzyme and/or functional fragment is obtained or derived from a marine bacterium selected from the group of Meptuniibacter caesariensis species strain MED92, Reinekea sp. strain MED297, Marinomonas sp. strain MED121, unnamed gammaproteobacterium strain HTCC2207 and Marinobacter sp. strain ELB17 and equivalents and synonymous species thereof.

In various embodiments, the FAR enzyme and/or functional fragment can be derived or obtained from a γ proteobacterium of the order Oceanospirillilales. In some embodiments, the FAR enzyme and/or functional fragment can be derived from or obtained from the Oceanospirillilales family Oceanospirillaceae. In certain embodiments, the FAR enzyme and/or functional fragment can be derived from or obtained from an Oceanospirillaceae genus, such as but not limited to Oceanobacter. In a particular embodiment, the FAR enzyme and/or functional fragment can be derived from or obtained from the Oceanobacter species strain RED65 and has an amino acid sequence that is at least 70% identical, at least 75% identical, at least 80% identical, at least 85% identical, at least 90% identical, at least 93% identical, at least 95% identical, at least 97% identical and/or at least 98% identical to SEQ ID NO: 3 or a functional fragment thereof. In another specific embodiment, the FAR enzyme for use in the methods disclosed herein comprises or consists of a sequence having 100% identity to the sequence of SED ID NO: 3 (“FAR_Ocs”). In other specific embodiments, the isolated FAR enzyme or functional fragment is obtained or derived from Oceanobacter kriegii. In still other specific embodiments, the isolated FAR enzyme or functional fragment is obtained or derived from Oceanobacter strain WH099.

In various embodiments, the FAR enzyme is from a marine bacterium and is selected from the group consisting of FAR_Hch (Hahella chejuensis KCTC 2396 GenBank YP_{—}436183.1); FAR_Mac (from marine Actinobacterium strain PHSC20C1), FAR_JVC (JCVI_ORF_{—}1096697648832, GenBank Accession No. EDD40059.1; from a marine metagenome), FAR_Fer (JCVI_SCAF_{—}1101670217388; from a marine bacterium found at a depth of 12 m in an upwelling in the area of Fernandina Island, the Galapagos Islands, Ecuador), FAR_Key (JCVI_SCAF_{—}1097205236585, from a marine bacterium found at a depth of 1.7 m off the coast of Key West Fla.), and FAR_Gal (JCVI_SCAF_{—}1101670289386, at a depth of 0.1 m at Isabella Island, Galapagos Islands, Ecuador). Approximate sequence identity to M. algicola DG893 (FAR_Maa) and Oceanobacter sp. RED65 (FAR_Ocs) is given in the Table 3.

TABLE 3

| | % Sequence Identity to | % Sequence Identity to |
|---|---|---|
| | FAR_Maa | FAR_Ocs |
| FAR Gene | (SEQ ID NO: 1) | (SEQ ID NO: 3) |
| FAR_Maa | 100 | 46 |
| FAR_Mac | 32 | 31 |
| FAR_Fer | 61 | 36 |
| FAR_Gal | 25 | 25 |
| FAR_JVC | 34 | 30 |
| FAR_Key | 32 | 30 |
| FAR_Maq | 78 | 45 |
| FAR_Hch | 54 | 47 |

In one particular embodiment, the FAR enzyme is isolated or derived from the marine bacterium FAR_Gal. In other embodiments, the FAR enzyme or functional fragment is isolated or derived from an organism selected from the group consisting of Vitis vinifera (GenBank Accession No. CAO22305.1 or CAO67776.1), Desulfatibacillum alkenivorans (GenBank Accession No. NZ_ABII01000018.1), Stigmatella aurantiaca (NZ_AAMD01000005.1) and Phytophthora ramorum (GenBank Accession No.: AAQX01001105.1). Also included are bfar from Bombyx mori (which encodes FAR enzyme polypeptide of SEQ ID NO: 4); hfar from H. sapiens, jjfar from Simmondsia chinensis, MS2 from Zea mays, MS2, FAR4, FAR6, or FER4 from Arabidopsis thaliana (e.g. FAR6 having Accession NP_{—}115529); mfar1 and mfar2 from Mus musculus.

In certain embodiments, a FAR enzyme or functional fragment thereof that is especially suitable for the production of fatty alcohols is identified by the presence of one or more domains, which are found in proteins with FAR activity. In various embodiments, the one or more domains is identified by multiple sequence alignments using hidden Markov models (“HMMs”) to search large collections of protein families, for example, the Pfam collection available at http:/pfam.sanger.ac.uk/. See R. D. Finn et al. (2008) Nucl. Acids Res. Database Issue 36:D281-D288.

In certain embodiments, the one or more protein domains by which FAR enzymes are identified belongs to a family of NAD binding domains found in the male sterility proteins of arabidopsis and drosophila, as well as in the fatty acyl reductase enzyme from the jojoba plant (JJFAR). See Aarts M G et al. (1997) Plant J. 12:615-623. This family of binding domains is designated “NAD_binding_{—}4” (PF07993; see http://pfam.sanger.ac.uk/family?acc=PF07993). In various embodiments, the NAD_binding_{—}4 domain is found near the N-terminus of the putative FAR enzyme. In various embodiments, the one or more protein domains by which enzymes with FAR activity are identified belongs to a family of domains known as a “sterile” domain (PF03015; see http://pfam.sanger.ac.uk/family?acc=PF03015), which are also found in the male sterility proteins of Arabidopsis species and a number of other organisms. See Aarts M G et al. (1997) Plant J. 12:615-623. In particular embodiments, the sterile domain is found near the C-terminus of the putative FAR enzyme. In certain specific embodiments, a FAR enzyme for use in the methods described herein is identified by the presence of at least one NAD_binding_{—}4 domain near the N-terminus and the presence of at least one sterile domain near the C-terminus.

In certain embodiments, the NAD_binding_{—}4 domain of the putative FAR enzyme has an amino acid sequence that is at least 10%, such as at least 15%, such as at least 20%, such as at least 25%, such as at least 30%, such as at least 35%, such as at least 40%, such as at least 45%, such as at least 50%, such as at least 60%, such as at least 70%, such as at least 80%, such as at least 85%, such as at least 90% or more identical to the amino acid sequence of a known NAD_binding_{—}4 domain. See, e.g. Aarts M G et al. (1997) Plant J. 12:615-623. In various embodiments, the sterile domain of the putative FAR enzyme has an amino acid sequence that is at least 10%, such as at least 15%, such as at least 20%, such as at least 25%, such as at least 30%, such as at least 35%, such as at least 40%, such as at least 45%, such as at least 50% or more identical to the amino acid sequence of a known sterile domain. See id.

In some embodiments, the NAD_binding_{—}4 domain of the putative FAR enzyme has an amino acid sequence that is at least 10%, such as at least 15%, such as at least 20%, such as at least 25%, such as at least 30%, such as at least 35%, such as at least 40%, such as at least 45%, such as at least 50%, such as at least 60%, such as at least 70%, such as at least 80%, such as at least 85%, such as at least 90% or more identical to the amino acid sequence of one or more example polypeptides that form the definition of the NAD_binding_{—}4 Pfam domain (PF07993). In certain embodiments, the sterile domain of the putative FAR enzyme has an amino acid sequence that is at least 10%, such as at least 15%, such as at least 20%, such as at least 25%, such as at least 30%, such as at least 35%, such as at least 40%, such as at least 45%, such as at least 50% or more identical to the amino acid sequence of one or more example polypeptides that form the definition of the sterile Pfam domain (PF03015). In various embodiments, the NAD_binding_{—}4 domain or the sterile domain of the putative FAR enzyme is identified by an E-value of 1×10⁻⁴ or less, such as an E-value of 1×10⁻⁵, such as an E-value of 1×10⁻¹⁰, such as an E-value of 1×10⁻¹⁵, such as an E-value of 1×10⁻²⁰, such as an E-value of 1×10⁻²⁵, such as an E-value of 1×10⁻³⁰ or lower. As used herein, the term E-value (expectation value) is the number of hits that would be expected to have a score equal or better than a particular hit by chance alone. Accordingly, the E-value is a criterion by which the significance of a database search hit can be evaluated (see, e.g. further information located at URL: pfam.sanger.ac.uk/help; or URL: www.csb.yale.edu/userguides/seq/hmmer/docs/node5.html).

The FAR enzymes described herein have not previously been recognized as FAR enzymes because of the low homology of the FAR coding sequences to the sequences coding for proteins with known FAR activity, such as the FAR enzymes from S. chinensis ((FAR Sim); GenBank Accession no. AAD38039.1; gi|5020215|gb|AAD38039.1|AF149917_{—}1 acyl CoA reductase [Simmondsia chinensis]—Plant Physiol. 2000 March; 122(3):635-44. “Purification of a jojoba embryo fatty acyl-coenzyme A reductase and expression of its cDNA in high erucic acid rapeseed,” Metz J G, Pollard M R, Anderson L, Hayes T R, Lassner M W. PMID: 10712526), B. mori ((FAR Bom); GenBank Accession no. BAC79425.1; gi|33146307|dbj|BAC79425.1| fatty-acyl reductase [Bombyx mori]; Proc Natl Acad Sci USA 2003 Aug. 5; 100(16):9156-61. Epub 2003 July 18. “Pheromone gland-specific fatty-acyl reductase of the silkmoth, Bombyx mori,” Moto K, Yoshiga T, Yamamoto M, Takahashi S, Okano K, Ando T. Nakata T. Matsumoto S. PMID: 12871998). Arabidopsis thaliana (GenBank Accession no. DQ446732.1 or NM_{—}115529.1: gi|91806527|gb|DQ446732.1|Arabidopsis thaliana clone pENTR221-At3g44560; gi|18410556|ref|NM_{—}115529.1| Arabidopsis thaliana male sterility protein, putative (AT3G56700); Plant Physiol. 2009 May 15; 166(8):787-96. Epub 2008 December 4. “Functional expression of five Arabidopsis fatty acyl-CoA reductase genes in Escherichia coli,” Doan T T, Carlsson A S, Hamberg M, Bülow L. Stymne S, Olsson P. PMID: 19062129) or Ostrinia scapulalis (GenBank Accession no. EU817405.1; gi|2100631381|gb|EU8 17405.1| Ostrinia scapulalis FAR-like protein XIII; Insect Biochem. Mol Biol. 2009 February; 39(2):90-5. Epub 2008 October 26 “Pheromone-gland-specific fatty-acyl reductase in the adzuki bean borer, Ostrinia scapulalis (Lepidoptera: Crambidae),” Antony B, Fujii T, Moto K, Matsumoto S, Fukuzawa M, Nakano R, Tatsuki S, Ishikawa Y.).

Pathway of FIG. 1 and FIG. 2, Steps C and D—Alternative Two-Enzyme Reduction of Reduction of Glutaconyl-CoA (or -ACP) to 5-Hydroxypent-3-Enoate Through 5-Oxopent-3-Enoate Aldehyde Intermediate

As an alternative to the pathway of FIG. 1 and FIG. 2, Step B, the conversion of glutaconyl-CoA (or -ACP) to 5-hydroxypent-3-enoate can be carried out by two enzymes in two steps. In FIG. 1 and FIG. 2, Step C an acyl-CoA (or -ACP) reductase reduces the glutaconyl-CoA (or -ACP) to the aldehyde intermediate 5-oxopent-3-enoate with the oxidation of a first equivalent of NAD(P)H cofactor. Then, in FIG. 1 and FIG. 2, Step D, an alcohol dehydrogenase or ketoreductase reduces the 5-oxopent-3-enoate to 5-hydroxypent-3-enoate with the oxidation of a second equivalent of NAD(P)H cofactor.

A number of acyl-CoA (or -ACP) reductase enzymes in class 1.2.1 are known to have the ability to reduce fatty acyl-CoA compounds to the corresponding fatty aldehydes, and are provided in Table 4.

TABLE 4

| EC Number | Enzyme Name |
|---|---|
| 1.2.1.44 | Cinnamoyl-CoA reductase |
| 1.2.1.50 | Long-chain-fatty acyl-CoA reductase |
| 1.2.1.75 | Malonyl-CoA reductase |
| 1.2.1.76 | Succinate-semialdehyde dehydrogenase (acylating) |
| 1.2.1.80 | Long-chain acyl-(acyl-carrier protein) reductase |
| 1.2.1.n2 | Fatty acyl-CoA reductase |

Specific exemplary fatty acyl-CoA reductase enzymes classes EC 1.2.1.50, EC 1.2.1.76 and EC1.2.1.n2 that could be used in the engineered pathway of FIG. 1 or FIG. 2, Step C are shown in Table 5.

TABLE 5

| Gene | Organism | UniProt id | GenBank id | GI Number |
|---|---|---|---|---|
| luxC | Photobacterium | Q03324 | CAA46274.1 | 45567 |
| | leiognathi | | | |
| sucD | Clostridium kluyveri | P38947 | AAA92341.7 | 347072 |
| acr1 | Acinetobacter sp. | Q6F7B8 | CAG70041.1 | 49532335 |
| FAR1 | Gallus gallus | Q5ZM72 | CAG31171.1 | 53127684 |
| FAR1 | Arabidopsis | Q39152 | AED93034.1 | 332005651 |
| | thaliana | | | |
| FAR2 | Arabidopsis | Q08891 | AEE75132.1 | 332641611 |
| | thaliana | | | |
| FAR3 | Arabidopsis | Q93ZB9 | AEE86278.1 | 332660878 |
| | thaliana | | | |
| FAR6 | Arabidopsis | B9TSP7 | AEE79553.1 | 332616032 |
| | thaliana | | | |
| FAR8 | Arabidopsis | Q1PEI6 | AEE77915.1 | 332644394 |
| | thaliana | | | |

There are numerous alcohol dehydrogenases/ketoreductase that have been well-studied functionally and structurally, including extensive engineering to provide enzymes having improved properties. Engineered ketoreductases having improved properties (e.g., increased activity, enantioselectivity, and/or thermostability) are described in the patent publications US 20080318295A1; US 20090093031A1; US 20090155863A1; US 20090162909A1; US 20090191605A1; US 20100055751A1; WO/2010/025238A2; WO/2010/025287A2; and US 20100062499A1: each of which are incorporated by reference herein. Exemplary enzymes of this class, either as the wild type or after enzyme engineering/evolution, which are capable of reducing fatty aldehydes to the corresponding alcohol are shown in Table 6:

TABLE 6

| | | | | GI |
|---|---|---|---|---|
| Gene | Organism | UniProt id | GenBank id | Number |
| adh | Thermoanaerobacter | P14941 | CAA46053.1 | 1771791 |
| | brockii | | | |
| sadh | Rhodococcus ruber | Q8KLT9 | CAD36475.1 | 21615553 |
| radh | Lactobacillus brevis | Q84EX5 | CAD66648.1 | 28400789 |
| adhR | Lactobacillus kefir | Q6WVP7 | AAP94029.1 | 33112056 |
| ADH1 | Kluyveromyces lactis | P20369 | CAG98731.1 | 49645159 |
| AOD1 | Candida boidinii | Q00922 | AAA34321.1 | 170820 |
| YADH1 | Saccharomyces | P00330 | AAA34410.1 | 171025 |
| | cerevisiae | | | |
| ADH-T | Bacillus | P12311 | BAA14411.1 | 216230 |
| | stearothermophilus | | | |
| yqhD | Escherichia coli | Q46856 | BAE77068.7 | 85675815 |
| | (strain K12) | | | |

In some embodiments of the present disclosure, a reductase enzyme of Table 5 or Table 6 naturally occurs in the host cell used to prepare the recombinant host cell capable of producing 1,3-butadiene. In such an embodiment, no further modification of the host cell is needed to provide expression of enzymes capable of the conversion of substrate to product as in Steps C or D of FIG. 1 and FIG. 2. In certain embodiments, a naturally occurring gene, or a natural homolog of such a gene, encoding an enzyme of Tables 5 or 6 can be used to heterologously transform a host cell which lacks such a gene, and/or has such a gene but the native gene expresses either too little or too much of the desired enzyme. Accordingly, heterologous transformation with a gene of Tables 5 or 6 can provide a recombinant host cell with an improved property (e.g., altered expression of a gene, altered concentration of a substrate and/or product due to use of a non-native gene in the pathway). In certain embodiments, an engineered version of a gene of Tables 5 or 6 can be used to transform a host cell to provide an enzyme capable of the conversion of substrate to product as in Steps C or D of FIG. 1 and FIG. 2, having an improved property (e.g., increased conversion of the specific 5-oxopent-3-enoate substrate as in Step D).

Pathway of FIG. 1, Step E

The conversion of 5-hydroxypent-3-enoate to 1,3-butadiene can carried out by a dehydratase enzyme that decarboxylates (i.e., through loss of CO₂) and dehydrates (i.e., through loss of H₂O) the substrate, 5-hydroxypent-3-enoate, either simultaneously or in a step-wise fashion. Two classes of enzymes having this activity are shown in Table 7.

TABLE 7

| EC Number | Enzyme Name |
|---|---|
| 4.2.1.51 | Prephenate dehydratase |
| 4.2.1.91 | Arogenate dehydratase |

Exemplary dehydratase enzymes in the classes EC 4.2.1.51 and EC 4.2.1.91 that could be used in preparing an engineered pathway of FIG. 1, Step E of the present disclosure are shown in Table 8.

TABLE 8

| Gene | Organism | UniProt id | GenBank id | GI Number |
|---|---|---|---|---|
| ADT1 | Arabidopsis thaliana | Q9SA96 | AAD30242.1 | 4835776 |
| ADT2 | Arabidopsis thaliana | Q9SSE7 | AEE74577.1 | 332641056 |
| ADT3 | Arabidopsis thaliana | Q9ZUY3 | AEC08050.1 | 330252956 |
| ADT4 | Arabidopsis thaliana | O22241 | AEE77939.1 | 332644418 |
| ADT5 | Arabidopsis thaliana | Q9FNJ8 | AED93055.1 | 332005672 |
| ADT6 | Arabidopsis thaliana | Q9SGD6 | AEE28265.1 | 332190144 |
| pheA | Escherichia coli O157:H7 | P0A9J9 | AAG57710.1 | 12517021 |
| pheA | Escherichia coli K12 | P0A9J8 | AAA24330.1 | 147175 |
| pheA | Methanocaldococcus jannaschii | Q58054 | AAB98631.1 | 1591349 |
| pheC | Pseudomonas aeruginosa | Q01269 | AAC08596.1 | 2997758 |

In some embodiments of the present disclosure, a dehydratase enzyme of Table 8 naturally occurs in the host cell used to prepare the recombinant host cell capable of producing 1,3-butadiene. In such an embodiment, no further modification of the host cell is needed to provide expression of an enzyme capable of the conversion of substrate to product of Step E of FIG. 1. In certain embodiments, a naturally occurring gene, or a natural homolog of such a gene, encoding an enzyme of Table 8 can be used to heterologously transform a host cell which lacks such a gene, and/or has such a gene but the native gene expresses either too little or too much of the desired enzyme. Accordingly, heterologous transformation with a gene of Table 8 can provide a recombinant host cell with an improved property (e.g., altered expression of a gene, altered concentration of a substrate and/or product due to use of a non-native gene in the pathway). In certain embodiments, an engineered version of a gene of Table 8 can be used to transform a host cell to provide an enzyme capable of the conversion of substrate to product of Step E of FIG. 1 having an improved property (e.g., increased conversion of the specific substrate of Step E).

Pathway of FIG. 2, Step E

The conversion of a hydroxyl group (e.g., as in an alcohol) to the corresponding phosphate ester is an ubiquitous reaction found in all organisms. Accordingly, there are a large number of alcohol kinase enzymes in class EC 2.7.1.x that are known to catalyze conversion of an alcohol to a phosphate as shown in Table 9.

TABLE 9

| EC Number | Enzyme name |
|---|---|
| EC 2.7.1.1 | hexokinase |
| EC 2.7.1.2 | glucokinase |
| EC 2.7.1.3 | ketohexokinase |
| EC 2.7.1.4 | fructokinase |
| EC 2.7.1.5 | rhamnulokinase |
| EC 2.7.1.6 | galactokinase |
| EC 2.7.1.7 | mannokinase |
| EC 2.7.1.8 | glucosamine kinase |
| EC 2.7.1.10 | phosphoglucokinase |
| EC 2.7.1.11 | 6-phosphofructokinase |
| EC 2.7.1.12 | gluconokinase |
| EC 2.7.1.13 | dehydrogluconokinase |
| EC 2.7.1.14 | sedoheptulokinase |
| EC 2.7.1.15 | ribokinase |
| EC 2.7.1.16 | ribulokinase |
| EC 2.7.1.17 | xylulokinase |
| EC 2.7.1.18 | phosphoribokinase |
| EC 2.7.1.19 | phosphoribulokinase |
| EC 2.7.1.20 | adenosine kinase |
| EC 2.7.1.21 | thymidine kinase |
| EC 2.7.1.22 | ribosylnicotinamide kinase |
| EC 2.7.1.23 | NAD+ kinase |
| EC 2.7.1.24 | dephospho-CoA kinase |
| EC 2.7.1.25 | adenylyl-sulfate kinase |
| EC 2.7.1.26 | riboflavin kinase |
| EC 2.7.1.27 | erythritol kinase |
| EC 2.7.1.28 | triokinase |
| EC 2.7.1.29 | glycerone kinase |
| EC 2.7.1.30 | glycerol kinase |
| EC 2.7.1.31 | glycerate kinase |
| EC 2.7.1.32 | choline kinase |
| EC 2.7.1.33 | pantothenate kinase |
| EC 2.7.1.34 | pantetheine kinase |
| EC 2.7.1.35 | pyridoxal kinase |
| EC 2.7.1.36 | mevalonate kinase |
| EC 2.7.1.39 | homoserine kinase |
| EC 2.7.1.40 | pyruvate kinase |
| EC 2.7.1.41 | glucose-phosphate phosphodismutase |
| EC 2.7.1.42 | riboflavin phosphotransferase |
| EC 2.7.1.43 | glucuronokinase |
| EC 2.7.1.44 | galacturonokinase |
| EC 2.7.1.45 | 2-dehydro-3-deoxygluconokinase |
| EC 2.7.1.46 | L-arabinokinase |
| EC 2.7.1.47 | D-ribulokinase |
| EC 2.7.1.48 | uridine kinase |
| EC 2.7.1.49 | hydroxymethylpyrimidine kinase |
| EC 2.7.1.50 | hydroxyethylthiazole kinase |
| EC 2.7.1.51 | L-fuculokinase |
| EC 2.7.1.52 | fucokinase |
| EC 2.7.1.53 | L-xylulokinase |
| EC 2.7.1.54 | D-arabinokinase |
| EC 2.7.1.55 | allose kinase |
| EC 2.7.1.56 | 1-phosphofructokinase |
| EC 2.7.1.58 | 2-dehydro-3-deoxygalactonokinase |
| EC 2.7.1.59 | N-acetylglucosamine kinase |
| EC 2.7.1.60 | N-acylmannosamine kinase |
| EC 2.7.1.61 | acyl-phosphate-hexose phosphotransferase |
| EC 2.7.1.62 | phosphoramidate-hexose phosphotransferase |
| EC 2.7.1.63 | polyphosphate-glucose phosphotransferase |
| EC 2.7.1.64 | inositol-kinase |
| EC 2.7.1.65 | scyllo-inosamine-kinase |
| EC 2.7.1.66 | undecaprenol kinase |
| EC 2.7.1.67 | 1-phosphatidylinositol 4-kinase |
| EC 2.7.1.68 | 1-phosphatidylinositol-4-phosphate 5-kinase |
| EC 2.7.1.69 | protein-Nπ-phosphohistidine-sugar phosphotransferase |
| EC 2.7.1.71 | shikimate kinase |
| EC 2.7.1.72 | streptomycin 6-kinase |
| EC 2.7.1.73 | inosine kinase |
| EC 2.7.1.74 | deoxycytidine kinase |
| EC 2.7.1.76 | deoxyadenosine kinase |
| EC 2.7.1.77 | nucleoside phosphotransferase |
| EC 2.7.1.78 | polynucleotide ′-hydroxyl-kinase |
| EC 2.7.1.79 | diphosphate-glycerol phosphotransferase |
| EC 2.7.1.80 | diphosphate-serine phosphotransferase |
| EC 2.7.1.81 | hydroxylysine kinase |
| EC 2.7.1.82 | ethanolamine kinase |
| EC 2.7.1.83 | pseudouridine kinase |
| EC 2.7.1.84 | alkylglycerone kinase |
| EC 2.7.1.85 | β-glucoside kinase |
| EC 2.7.1.86 | NADH kinase |
| EC 2.7.1.87 | streptomycin ″-kinase |
| EC 2.7.1.88 | dihydrostreptomycin-6-phosphate 3′α-kinase |
| EC 2.7.1.89 | thiamine kinase |
| EC 2.7.1.90 | diphosphate-fructose-6-phosphate 1-phosphotransferase |
| EC 2.7.1.91 | sphinganine kinase |
| EC 2.7.1.92 | 5-dehydro-2-deoxygluconokinase |
| EC 2.7.1.93 | alkylglycerol kinase |
| EC 2.7.1.94 | acylglycerol kinase |
| EC 2.7.1.95 | kanamycin kinase |
| EC 2.7.1.100 | S-methyl-5-thioribose kinase |
| EC 2.7.1.101 | tagatose kinase |
| EC 2.7.1.102 | hamamelose kinase |
| EC 2.7.1.103 | viomycin kinase |
| EC 2.7.1.105 | 6-phosphofructo-2-kinase |
| EC 2.7.1.106 | glucose-,-bisphosphate synthase |
| EC 2.7.1.107 | diacylglycerol kinase |
| EC 2.7.1.108 | dolichol kinase |
| EC 2.7.1.113 | deoxyguanosine kinase |
| EC 2.7.1.114 | AMP-thymidine kinase |
| EC 2.7.1.118 | ADP-thymidine kinase |
| EC 2.7.1.119 | hygromycin-B 7″-O-kinase |
| EC 2.7.1.121 | phosphoenolpyruvate-glycerone phosphotransferase |
| EC 2.7.1.122 | xylitol kinase |
| EC 2.7.1.127 | inositol-trisphosphate 3-kinase |
| EC 2.7.1.130 | tetraacyldisaccharide 4′-kinase |
| EC 2.7.1.134 | inositol-tetrakisphosphate 1-kinase |
| EC 2.7.1.136 | macrolide 2′-kinase |
| EC 2.7.1.137 | phosphatidylinositol 3-kinase |
| EC 2.7.1.138 | ceramide kinase |
| EC 2.7.1.140 | inositol-tetrakisphosphate 5-kinase |
| EC 2.7.1.142 | glycerol-3-phosphate-glucose phosphotransferase |
| EC 2.7.1.143 | diphosphate-purine nucleoside kinase |
| EC 2.7.1.144 | tagatose-6-phosphate kinase |
| EC 2.7.1.145 | deoxynucleoside kinase |
| EC 2.7.1.146 | ADP-dependent phosphofructokinase |
| EC 2.7.1.147 | ADP-dependent glucokinase |
| EC 2.7.1.148 | 4-(cytidine 5′-diphospho)-2-C-methyl-D-erythritol kinase |
| EC 2.7.1.149 | 1-phosphatidylinositol-5-phosphate 4-kinase |
| EC 2.7.1.150 | 1-phosphatidylinositol-3-phosphate 5-kinase |
| EC 2.7.1.151 | inositol-polyphosphate multikinase |
| EC 2.7.1.153 | phosphatidylinositol-4,5-bisphosphate 3-kinase |
| EC 2.7.1.154 | phosphatidylinositol-4-phosphate 3-kinase |
| EC 2.7.1.156 | adenosylcobinamide kinase |
| EC 2.7.1.157 | N-acetylgalactosamine kinase |
| EC 2.7.1.158 | inositol-pentakisphosphate-kinase |
| EC 2.7.1.159 | inositol-1,3,4-trisphosphate 5/6-kinase |
| EC 2.7.1.160 | 2′-phosphotransferase |
| EC 2.7.1.161 | CTP-dependent riboflavin kinase |
| EC 2.7.1.162 | N-acetylhexosamine 1-kinase |
| EC 2.7.1.163 | hygromycin B 4-O-kinase |
| EC 2.7.1.164 | O-phosphoseryl-tRNASec kinase |
| EC 2.7.1.165 | glycerate-kinase |
| EC 2.7.1.166 | 3-deoxy-D-manno-octulosonic acid kinase |
| EC 2.7.1.167 | D-glycero-β-D-manno-heptose-7-phosphate kinase |
| EC 2.7.1.168 | D-glycero-α-D-manno-heptose-7-phosphate kinase |
| EC 2.7.1.169 | pantoate kinase |
| EC 2.7.1.170 | anhydro-N-acetylmuramic acid kinase |
| EC 2.7.1.171 | protein-fructosamine 3-kinase |
| EC 2.7.1.172 | protein-ribulosamine 3-kinase |

In particular, based on their known activity and structure, the alcohol kinase enzymes in classes EC 2.7.1.30, EC 2.7.1.32, EC 2.7.1.36, EC 2.7.1.39 and EC 2.7.1.82 are well-suited for converting 5-hydroxypent-3-enoate to the corresponding phosphate compound, 5-(phosphonatooxy)pent-3-enoate as FIG. 2, Step E. Some exemplary alcohol kinases include glycerol kinase (EC 2.7.1.30; J. Biol. Chem. 1955, 211, 951), choline kinase (EC 2.7.1.32; J. Biol. Chem. 1953, 202, 431), mevalonate kinase (EC 2.7.1.36; J. Biol. Chem. 1958, 233, 1100), homoserine kinase (EC 2.7.1.39; J. Biochem. 1957, 44, 299), ethanolamine kinase (EC 2.7.1.82; Biochim. Biophys. Acta. 1972, 276, 143). Additionally, phosphorylation of simple alcohols by bacterial (S. felxneri and S. enterica) non-specific acid phosphatases (UniProt Q71EB8) has been demonstrated (Adv. Synth. Catal. 2005, 347, 1155). Also, it has been reported that isopentyl phosphate kinase from peppermint (Mentha×piperita) which normally phosphorylates isopentyl phosphate to the corresponding pyrophosphate also has activity on converting isopentenol and dimethylallyl alcohol to the corresponding phosphate (PNAS 1999, 96, 13714). These and other exemplary alcohol kinase enzymes from these classes that could be used in preparing an engineered pathway of FIG. 2, Step E of the present disclosure are shown in Table 10.

TABLE 10

| Gene | Organism | UniProt id | GenBank id | GI Number |
|---|---|---|---|---|
| GUT1 | Saccharomyces cerevisiae | P32190 | CAA48791.1 | 312423 |
| glpK | Escherichia coli (strain K12) | P0A6F3 | AAA23913.1 | 142660 |
| CHKA | Homo sapiens | P35790 | BAA01547.1 | 219541 |
| Chka | Mus musculus | O54804 | BAA88153.1 | 6539495 |
| Chkb | Mus musculus | O55229 | BAA24891.1 | 2897731 |
| ckb-2 | Caenorhabditis elegans | P46559 | CAA84301.2 | 29603337 |
| CKI1 | Saccharomyces cerevisiae | P20485 | AAA34499.1 | 171231 |
| MVK | Homo sapiens | Q03426 | AAF82407.1 | 9049533 |
| mvk | Dictyostelium discoideum | Q86AG7 | EAL71443.1 | 60472399 |
| mvk | Methanocaldococcus jannaschii | Q58487 | AAB99088.1 | 1591731 |
| Mvk | Rattus norvegicus | P17256 | AAA41588.1. | 205378 |
| ERG12 | Saccharomyces cerevisiae | P07277 | CAA39359.1 | 3684 |
| mk | Arabidopsis thaliana | P46086 | AAD31719.1 | 4883990 |
| THR1 | Saccharomyces cerevisiae | P17423 | AAA34154.1 | 172978 |
| thrB | Escherichia coli (strain K12) | P00547 | AAA50618.1 | 529240 |
| thrB | Methanocaldococcus jannaschii | Q58504 | AAB99107 | 1591748 |

In some embodiments of the present disclosure, an alcohol kinase enzyme of Table 10 naturally occurs in the host cell used to prepare the recombinant host cell capable of producing 1,3-butadiene. In such an embodiment, no further modification of the host cell is needed to provide expression of an enzyme capable of the conversion of substrate to product of FIG. 2, Step E. In certain embodiments, a naturally occurring gene, or a natural homolog of such a gene, encoding an enzyme of Table 10 can be used to heterologously transform a host cell which lacks such a gene, and/or has such a gene but the native gene expresses either too little or too much of the desired enzyme. Accordingly, heterologous transformation with a gene of Table 10 can provide a recombinant host cell with an improved property (e.g., altered expression of a gene, altered concentration of a substrate and/or product due to use of a non-native gene in the pathway). In certain embodiments, an engineered version of a gene of Table 10 can be used to transform a host cell to provide an enzyme capable of the conversion of substrate to product of FIG. 2, Step E having an improved property (e.g., increased conversion of the substrate, 5-hydroxypent-3-enoate to the product, 5-(phosphonatooxy)pent-3-enoate as in FIG. 2, Step E).

Pathway of FIG. 2, Step F

The phosphate product of FIG. 2, Step E, 5-(phosphonatooxy)pent-3-enoate, is converted to the desired product 1,3-butadiene via the elimination of a phosphate group with concomitant decarboxylation, as in FIG. 2, Step F. Generally, phosphate elimination is catalyzed by phosphate lyase enzymes in class EC 4.1.1.x. Mevalonate pyrophosphate decarboxylase (EC 4.1.1.33) catalyzes the similar reaction and has been shown to have promiscuous activity (e.g. the presence of the pyrophosphate moiety is not necessary: Appl. Environ. Microbiol. 2010, 76, 8004). Exemplary diphosphomevalonate decarboxylase enzymes (EC 4.1.1.33) are shown in Table 11.

TABLE 11

| Gene | Organism | UniProt id | GenBank id | GI Number |
|---|---|---|---|---|
| MVD | Homo sapiens | P53602 | EAW66792.1 | 119587196 |
| MVD1 | Saccharomyces | P32377 | CAA66158 | 1292890 |
| | cerevisiae | | | |
| Mvd | Mus musculus | Q99JFA | CAC35731 | 13539580 |
| mvaD | Streptococcus | Q9A097 | AAK33797.1 | 13622042 |
| | pygenes serotype | | | |
| | M1 | | | |

In some embodiments, a naturally occurring gene, such as a homolog of a gene in Table 11, having a phosphate elimination activity capable of converting 5-(phosphonatooxy)pent-3-enoate to 1,3-butadiene can be identified. Such a gene can then be used to heterologously transform a host cell which lacks this gene, and/or has such a gene but the native activity is not sufficient. Accordingly, heterologous transformation with a homolog of a gene of Table 12 can provide a recombinant host cell with an improved property (e.g., altered expression of a gene, altered concentration of a substrate and/or product due to use of a non-native gene in the pathway).

In some embodiments, an engineered version of a gene of Table 11, or a engineered version of a homolog of a gene of Table 11, can be used to transform a host cell to provide an enzyme capable of the conversion of substrate to product of FIG. 2, Step F having an improved property (e.g., increased conversion of the substrate 5-(phosphonatooxy)pent-3-enoate to 1,3-butadiene).

### 6.3. HOST CELL SELECTION AND ENGINEERING

In some embodiments, the present disclosure provides a method for producing a recombinant host cell, wherein the method comprises transforming a suitable host cell with one or more polynucleotides or nucleic acid constructs encoding: (a) a carboxylase enzyme, wherein the enzyme is capable of converting crotonyl-CoA (or -ACP) to glutaconyl-CoA (or -ACP); (b) a FAR enzyme, wherein the enzyme is capable of converting glutaconyl-CoA (or -ACP) to 5-hydroxypent-3-enoate; (c) a dehydratase enzyme capable of converting 5-hydroxypent-3-enoate to 1,3-butadiene; (d) a kinase enzyme capable of converting 5-hydroxypent-3-enoate to 5-(phosphonatooxy)pent-3-enoate; and/or (e) a decarboxylase enzyme capable of converting 5-(phosphonatooxy)pent-3-enoate to 1,3-butadiene. In some embodiments, the method comprises transforming the suitable host cell with one or more nucleic acid constructs encoding acyl-CoA reductase and an alcohol dehydrogenase capable of converting glutaconyl-CoA (or -ACP) to 5-hydroxypent-3-enoate via formation of a 5-oxopent-3-enoate intermediate.

In some embodiments, the host cell is a bacterial cell. In some embodiments, the host cell is a yeast cell. The transformed or transfected host cell is cultured in a suitable nutrient medium under conditions permitting the expression of the carboxylase enzyme of FIG. 1 or 2, Step A, the FAR enzyme of FIG. 1 or 2, Step B, the acyl-CoA reductase and alcohol dehydrogenase of FIG. 1 or 2, Steps C and D, the dehydratase enzyme of FIG. 1, Step E, the kinase enzyme of FIG. 2, Step E, and/or the decarboxylase enzyme of FIG. 2, Step F. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection).

A. Host Cells

The recombinant host cells of the present invention generally comprise a recombinant polynucleotide encoding one or more enzymes selected from the engineered pathways of FIG. 1 or 2, such as: (a) a carboxylase enzyme, wherein the enzyme is capable of converting crotonyl-CoA (or -ACP) to glutaconyl-CoA (or -ACP): (b) a FAR enzyme, wherein the enzyme is capable of converting glutaconyl-CoA (or -ACP) to 5-hydroxypent-3-enoate; (c) a dehydratase enzyme capable of converting 5-hydroxypent-3-enoate to 1,3-butadiene; (d) a kinase enzyme capable of converting 5-hydroxypent-3-enoate to 5-(phosphonatooxy)pent-3-enoate; and/or (e) a decarboxylase enzyme capable of converting 5-(phosphonatooxy)pent-3-enoate to 1,3-butadiene. Suitable host cells include, but are not limited to microorganisms including bacteria, yeast, filamentous fungi and algae. In certain embodiments, microorganisms useful as recombinant host cells are wild-type microorganisms. In certain embodiments, host cell is the bacteria Escherichia coli. In some embodiments, the host is a the yeast, and in particular embodiments, an oleaginous yeast.

In various embodiments, microorganisms useful as recombinant host cells are genetically modified. As used herein, “genetically modified” microorganisms include microorganisms having one or more endogenous genes removed, microorganisms having one or more endogenous genes with reduced expression compared to the parent or wild-type microorganism, or microorganisms having one or more genes overexpressed compared to the parent or wild-type microorganism. In certain embodiments, the one or more genes that are overexpressed are endogenous to the microorganism. In some embodiments, the one or more genes that are overexpressed are heterologous to the microorganism.

In certain embodiments, the genetically modified microorganism comprises an inactivated or silenced endogenous gene that codes for a protein involved in the biosynthesis of fatty acyl-CoA substrates. In particular embodiments, the inactive or silenced gene encodes a fatty acyl-ACP thioesterase or a fatty acyl-CoA synthetase (FACS).

In certain embodiments, the genetically modified microorganism alters (i.e., increases or decreases) the expression a gene that encodes one or more of the enzymes in the pathway of enzymes of FIG. 1 and FIG. 2, and/or a gene that encodes one or more proteins other than the enzymes in the pathway of enzymes of FIG. 1 and FIG. 2. In various embodiments, the altered expression of the one or more proteins can alter the rate at which the recombinant cell produces or metabolizes any of the compounds in the pathways of FIG. 1 and/or FIG. 2. In some embodiments, the one or more genes having altered expression encode enzymes directly involved in host cell metabolism of substrates or products of the engineered pathways of FIG. 1 and/or FIG. 2. In some embodiments, the gene having altered expression is endogenous to the host cell. In other embodiments, the gene having altered expression is heterologous to the host cell.

B. Prokaryotic Host Cells

In some embodiments, the host cell is a prokaryotic cell. Suitable prokaryotic cells include gram positive, gram negative and gram-variable bacterial cells. In certain embodiments, host cells include, but are not limited to, species of a genus selected from the group consisting of Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Cyanobacteria, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmun, Streptomyces, Streptococcus, Synnecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In particular embodiments, the host cell is a species of a genus selected from the group consisting of Agrobacterium, Arthrobacter, Bacillus, Clostridium, Corynebacterium, Escherichia, Erwinia, Geobacillus, Klebsiella, Lactobacillus, Mycobacterium, Pantoea, Rhodococcus, Streptomyces and Zymomonas.

In particular embodiments, the bacterial host cell is a species of the genus Escherichia, e.g., E. coli. E. coli provides a good prokaryotic microorganism for producing a recombinant host cell capable of producing 1,3-butadiene under aerobic, anaerobic or microaerobic conditions. Examples of chemicals produced by recombinant E. coli host cells include ethanol, lactic acid, succinic acid, and the like. In certain embodiments, the E. coli is a wild-type bacterium. In various embodiments, the wild-type E. coli bacterial strain useful in the processes described herein is selected from, but not limited to, strain W3110, strain MG1655 and strain BW25113. In other embodiments, the E. coli is genetically modified. Examples of genetically modified E. coli useful as recombinant host cells include, but are not limited to, genetically modified E. coli found in the Keio Collection, available from the National BioResource Project at NBRP E. coli, Microbial Genetics Laboratory, National Institute of Genetics 1111 Yata, Mishima, Shizuoka, 411-8540.

In particular embodiments, the genetically modified E. coli comprises an inactivated or silenced endogenous fadD gene, which codes for an acyl-CoA synthetase protein. In other embodiments the genetically modified E. coli comprises an inactivated of silenced endogenous fadK gene, which codes for an endogenous short-chain acyl-CoA synthetase. In still other embodiments, the genetically modified E. coli comprises an inactivated or silenced endogenous fadD gene and an inactivated or silenced endogenous fadK gene. In other embodiments, the genetically modified E. coli comprises an endogenous fadD gene that has reduced expression compared to the parent or wild-type strain. In various embodiments, the genetically modified E. coli comprises an endogenous fadK gene that has reduced expression compared to the parent or wild-type strain.

In certain embodiments, the recombinant host cell is an industrial bacterial strain. Numerous bacterial industrial strains are known and suitable for use in the methods disclosed herein. In some embodiments, the bacterial host cell is a species of the genus Bacillus, e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell is a species of the genus Bacillus and is selected from the group consisting of B. subtilis, B. pumilus, B. licheniformis, B. clausii, B. stearothermophilus, B. megaterium and B. amyloliquefaciens.

In some embodiments the bacterial host cell is a species of the genus Erwinia, e.g. E. uredovora, E. carotovora, E. ananas, E. herbicola, E. punctata or E. terreus.

In other embodiments the bacterial host cell is a species of the genus Pantoea, e.g., P. citrea or P. agglomerans.

In still other embodiments, the bacterial host cell is a species of the genus Streptomyces, e.g., S. ambofaciens, S. achromogenes, S. avermitilis, S. coelicolor, S. aureofaciens, S. aureus, S. fungicidicus, S. griseus or S. lividans.

In further embodiments, the bacterial host cell is a species of the genus Zymomonas, e.g., Z. mobilis or Z. lipolytica.

In further embodiments, the bacterial host cell is a species of the genus Rhodococcus, e.g. R. opacus.

C. Yeast Best Cells

In certain embodiments, the recombinant host cell is a yeast. In various embodiments, the yeast host cell is a species of a genus selected from the group consisting of Candida, Hansenula, Saccharomyces, Schizosaccharomyces, Pichia, Kluyveromyces, and Yarrowia. In particular embodiments, the yeast host cell is a species of a genus selected from the group consisting of Saccharomyces, Candida, Pichia and Yarrowia.

In various embodiments, the yeast host cell is selected from the group consisting of Hansenula polymorpha, Saccharomyces cerevisiae, Saccaromyces carlsbergensis, Saccharomyces diastaicus, Saccharomyces norbensis, Saccharomyces kluyveri, Schizosaccharomyces pombe, Pichia pastoris, Pichia finlandica, Pichia trehalophila, Pichia ferniemtans, Issatchenkia orientalis, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, Candida krusei, Candida ethanolic and Yarrowia lipolytica and synonyms or taxonomic equivalents thereof.

In certain embodiments, the yeast host cell is a wild-type cell. In various embodiments, the wild-type yeast cell strain is selected from, but not limited to, strain BY4741, strain FL100a, strain INVSC1, strain NRRL Y-390, strain NRRL Y-1438, strain NRRL YB-1952, strain NRRL Y-5997, strain NRRL Y-7567, strain NRRL Y-1532, strain NRRL YB-4149 and strain NRRL Y-567. In other embodiments, the yeast host cell is genetically modified. Examples of genetically modified yeast useful as recombinant host cells include, but are not limited to, genetically modified yeast found in the Open Biosystems collection found at the following URL: www.openbiosystems.com/GeneExpression/Yeast/YKO/ (see also e.g., Winzeler et al. (1999) Science 285:901-906).

In other embodiments, the recombinant host cell is an oleaginous yeast. Oleaginous yeast are organisms that accumulate lipids such as tri-acylglycerols. Examples of oleaginous yeast include, but are not limited to, organisms selected from the group consisting of Yarrowia lipolytica, Yarrowia paralipolytica, Candida revkaufi, Candida pulcherrima, Candida tropicalis, Candida utilis, Candida curvala D, Candida curvala R, Candida diddensiae, Candida boldinii, Rhodotorula glutinous, Rhodotorula graminis, Rhodotorula mucilaginosa, Rhodotorula minuta, Rhodotorula bacarum, Rhodosporidium toruloides, Cryptococcus (terricolus) albidus var. albidus, Cryptococcus laurentii, Trichosporon pullans, Trichosporon cutaneum, Trichosporon cutancum, Trichosporon pullulans, Lipomyces starkeyii, Lipomyces lipoferus, Lipomyces tetrasporus, Endomyropsis vernalis, Hansenula ciferri, Hansenula saturnus, and Trigonopsis variables. In particular embodiments, the oleaginous yeast is Y. lipolytica. In certain embodiments, Yarrowia lipolytica strains include, but are not limited to, DSMZ 1345, DSMZ 3286, DSMZ 8218, DSMZ 70561, DSMZ 70562, and DSMZ 21175.

In certain embodiments, the oleaginous yeast is a wild-type organism. In other embodiments, the oleaginous yeast is genetically modified.

In yet other embodiments, the recombinant host cell is a filamentous fungus. In certain embodiments, the filamentous fungal host cell is a species of a genus selected from the group consisting of Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, Volvariella, and teleomorphs, synonyms or taxonomic equivalents thereof.

In some embodiments, the filamentous fungal host cell is an Aspergillus species, a Chrysosporium species, a Corynascus species, a Fusarium species, a Humicola species, a Myceliophthora species, a Neurospora species, a Penicillum species, a Tolypocladium species, a Tramates species, or Trichoderma species. In other embodiments, the Trichoderma species is selected from T. longibrachiatum, T. viride, Hypocrea jecorina and T. reesei; the Aspergillus species is selected from A. awamori, A. funigatus, A. japonicus, A. nidulans, A. niger, A. aculeatus, A. foetidus, A. oryzae, A. sojae, and A. kawachi; the Chrysosporium species is C. lucknowense; the Fusarium species is selected from F. graminum, F. oxysporum and F. venenatum; the Myceliophthora species is M. thermophilia; the Neurospora species is N. crassa; the Humicola species is selected from H. insolens, H. grisea, and H. lanuginosa; the Penicillum species is selected from P. purpurogenum, P. chrysogenum, and P. verruculosum; the Thielavia species is T. terrestris; and the Trametes species is selected from T. villosa and T. versicolor.

In some embodiments, the filamentous fungal host is a wild-type organism. In other embodiments, the filamentous fungal host is genetically modified.

In certain particular embodiments, recombinant host cells for use in the methods described herein are derived from strains of Escherichia coli, Bacillus, Saccharomyces, Streptomyces and Yarrowia.

In certain embodiments the host cell is a Yarrowia cell, such as a Y. lipolytica cell.

Cells which are useful in the practice of the present disclosure include prokaryotic and eukaryotic cells which are readily accessible from a number of culture collections and other sources, e.g., the American Type Culture Collection (ATCC), Deutsche Sammlung von Mikroorganismen und Zellkulturen GmbH (DSMZ) (German Collection of Microorganisms and Cell Culture), Centraalbureau Voor Schimmelcultures (CBS), and Agricultural Research Service Patent Culture Collection, Northern Regional Research Center (NRRL). Yarrowia lipolytica is available, as a non-limiting example, from the ATCC under accession numbers 20362, 18944, and 76982.

In some embodiments, the recombinant host cell comprising a polynucleotide encoding a FAR enzyme described herein, further lacks a gene encoding a fatty acyl-CoA synthetase (FACS) and/or a gene encoding a fatty acyl-ACP thioesterase (TE). Without being bound to a particular theory, 5-hydroxypent-3-enoate, and subsequent 1,3-butadiene production may be increased in a recombinant host cell lacking a gene encoding a FACS and/or a TE because silencing or inactivating the FACS and/or TE gene may inactivate a competing biosynthetic pathways. Accordingly, in some embodiments, the recombinant E. coli host cells of the present disclosure can further comprise a silenced or inactivated fatty acyl-CoA synthetase fadD gene and/or silenced or inactivated short chain fatty acyl-CA synthetase fadK gene. The recombinant E. coli host can be genetically modified to be silenced or inactivated in one or more of the additional genes described above.

D. Host Cell Transformation and Culture

Recombinant polynucleotides of the disclosure, e.g. polynucleotides encoding a FAR enzyme, may be introduced into host cells for expression of the FAR enzyme in the engineered pathway of FIG. 1 and/or FIG. 2. In some embodiments, the recombinant polynucleotide may be introduced into the cell as a self-replicating episome (e.g., expression vector) or may be stably integrated into the host cell DNA.

In some embodiments, a host cell is transformed with a recombinant polynucleotide encoding an enzyme in an engineered pathway of FIG. 1 and/or FIG. 2. In transformation, the recombinant polynucleotide that is introduced into the host cell remains in the genome or on a plasmid or other stably maintained vector in the cell and is capable of being inherited by the progeny thereof. Stable transformation is typically accomplished by transforming the host cell with an expression vector comprising the polynucleotide of interest (e.g. the polynucleotide encoding a FAR enzyme) along with a selectable marker gene (e.g., a gene that confers resistance to an antibiotic). Only those host cells which have integrated the polynucleotide sequences of the expression vector into their genome will survive selection with the marker (e.g., antibiotic). These stably transformed host cells can then be propagated according to known methods in the art.

Methods, reagents and tools for transforming host cells described herein, such as bacteria (include E. coli), yeast (including oleaginous yeast) and filamentous fungi are known in the art. General methods, reagents and tools for transforming, e.g., bacteria can be found, for example, in Sambrook et al (2001) Molecular Cloning: A Laboratory Manual, 3^{rd} ed., Cold Spring Harbor Laboratory Press, New York. Methods, reagents and tools for transforming yeast are described in “Guide to Yeast Genetics and Molecular Biology,” C. Guthrie and G. Fink, Eds., Methods in Enzymology 350 (Academic Press, San Diego, 2002). Methods, reagents and tools for transforming, culturing, and manipulating Y. lipolytica are found in “Yarrowia lipolytica,” C. Madzak, J. M. Nicaud and C. Gaillardin in “Production of Recombinant Proteins. Novel Microbial and Eucaryotic Expression Systems,” G. Gellissen. Ed. 2005, which is incorporated herein by reference for all purposes. In some embodiments, introduction of the DNA construct or vector of the present disclosure into a host cell can be effected by calcium phosphate transfection, DEAE-Dextran mediated transfection, PEG-mediated transformation, electroporation, or other common techniques (See Davis et al., 1986, Basic Methods in Molecular Biology, which is incorporated herein by reference).

The recombinant host cells can be cultured in conventional nutrient media modified as appropriate for activating promoters, selecting transformants, or amplifying the expression of certain pathway enzymes (e.g., the FAR enzyme of FIG. 1 or 2, Step B). Culture conditions, such as temperature, pH and the like, are those previously used with the host cell selected for expression, and will be apparent to those skilled in the art. As noted, many references are available for the culture and production of many cells, including cells of bacterial, plant, animal (especially mammalian) and archaeobacterial origin. See e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons. Inc. New York. N.Y.; Gamborg and Phillips (eds) (1995) Plant Cell, Tissue and Organ Culture; Fundamental Methods Springer Lab Manual, Springer-Verlag (Berlin Heidelberg New York); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford. U.K. ISBN 0 12 198370 6, all of which are incorporated herein by reference. Media for host cell culture in general are set forth in Atlas and Parks (eds.) The Handbook of Microbiological Media (1993) CRC Press, Boca Raton, Fla., which is incorporated herein by reference. Additional information for host cell culture is found in available commercial literature such as the Life Science Research Cell Culture Catalogue (1998) from Sigma-Aldrich. Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference.

### 6.4. METHODS OF USING THE RECOMBINANT HOST CELLS FOR PRODUCING 1,3-BUTADIENE

A. Biosynthetic Production and Isolation of 1,3-Butadiene

The present disclosure also provides methods for producing 1,3-butadiene by fermentation of the recombinant host cells comprising one or more recombinant polynucleotides as described herein. As noted elsewhere herein, in some embodiments, the recombinant host cells comprise an engineered pathway of enzymes of FIG. 1, that provides enzymes capable of producing 1,3-butadiene biosynthetically in three steps (FIG. 1, Steps A, B, and E) from crotonyl-CoA (or -ACP) via glutaconyl-CoA (or -ACP) and 5-hydroxypent-3-enoate intermediates. In other embodiments, the recombinant host cells comprise an engineered pathway of enzymes of FIG. 2, that provides enzymes capable of producing 1,3-butadiene biosynthetically in four steps (FIG. 2, Steps A, B, E, and F) from crotonyl-CoA (or -ACP) via glutaconyl-CoA (or -ACP), 5-hydroxypent-3-enoate, and 5-(phosphonatooxy)pent-3-enoate intermediates. The same general methods for producing a fermentation product can be used with the recombinant host cells comprising an engineered pathway of either FIG. 1 or FIG. 2. Accordingly, in some embodiments the present disclosure provides a method of producing 1,3-butadiene, wherein the method comprises: (a) providing the recombinant host cell as described herein; (b) providing a fermentation medium comprising a fermentable sugar, (c) contacting the fermentation medium with the recombinant host cell under conditions suitable for generating 1,3-butadiene; and optionally (d) recovering the 1,3-butadiene.

Generally, in the embodiments of the methods for producing the 1,3-butadiene fermentation product described above and elsewhere herein, the fermentable sugar may comprise products of a cellulosic saccharification process, including, for example, mono-, di-, and trisaccharides (e.g., glucose, xylose, sucrose, maltose, and the like), and more complex polysaccharide carbohydrates (e.g., lignocellulose, xylans, cellulose, starch, and the like), and the like. Compositions of fermentation media suitable for the growth of recombinant host cells such as E. coli, yeast, and filamentous fungi are well known in the art. See, for example, Yeast Protocols (1^{st} and 2^{nd} edition), Hahan-Hagerdal Microbial Cell Factories 2005, Walker Adv. In Applied Microbiology (2004), which is incorporated herein by reference.

Fermentation conditions suitable for generating the desired fermentation product, 1,3-butadiene, are well known in the art. The suitable conditions can comprise aerobic, microaerobic or anaerobic conditions. In some embodiments, the suitable conditions for fermentation can comprise anaerobic conditions. Typical anaerobic conditions are the absence of oxygen (i.e., no detectable oxygen), or less than about 5, about 2.5, or about 1 mmol/L/h oxygen. In the absence of oxygen, the NADH produced in glycolysis cannot be oxidized by oxidative phosphorylation. Under anaerobic conditions, pyruvate or a derivative thereof may be utilized by the host cell as an electron and hydrogen acceptor in order to generate NAD⁺. In certain embodiments of the present disclosure, when the fermentation process is carried out under anaerobic conditions, pyruvate may be reduced to a fermentation product such as ethanol butanol, or lactic acid.

Typically, the suitable conditions comprise running the fermentation at a temperature that is optimal for the recombinant host cell. For example, the fermentation process may be performed at a temperature in the range of from about 25° C. to about 42° C. Typically the process is carried out a temperature that is less than about 38° C., less than about 35° C., less than about 33° C., less than about 38° C., but at least about 20° C., 22° C. or 25° C.

In some embodiments of the methods, the recombinant host cells of the present disclosure are grown under batch or continuous fermentation conditions. Classical batch fermentation is a closed system, wherein the composition of the medium is set at the beginning of the fermentation and is not subject to artificial alterations during the fermentation. A variation of the batch system is a fed-batch fermentation which also finds use in the present disclosure. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art.

Continuous fermentation is carried out using an open system where a defined fermentation generally maintains the culture at a constant high density where cells are primarily in log phase growth. Continuous fermentation systems strive to maintain steady state growth conditions. Methods for modulating nutrients and growth factors for continuous fermentation processes as well as techniques for modulating nutrients and growth factors for continuous fermentation processes as well as techniques for maximizing the rate of product formation are well known in the art of industrial microbiology.

### 7. EXAMPLES

Various features and embodiments of the disclosure are illustrated in the following representative examples, which are intended to be illustrative, and not limiting.

### Example 1

### Recombinant Host Cell with an Engineered Pathway for Production of 1,3-Butadiene Via Glutaconyl-CoA and 5-Hydroxypent-3-enoate Intermediates

This Example illustrates the preparation of a recombinant E. coli host cell that expresses the genes in the engineered pathways of FIG. 1 for the production of 1,3-butadiene from fermentable sugar.

The following genes of the engineered pathway of FIG. 1, Steps A, B, and E are synthesized: (1) the wild type or an engineered variant of Glycine max gene MCCA (Uniprot Q42777) encoding 3-methylcrotonyl-CoA carboxylase (EC 6.4.1.4) which is capable of converting crotonyl-CoA to glutaconyl-CoA; (2) an engineered variant of FAR enzyme (EC 1.1.1*) derived from Marinobacter algicola DG893 gene FAR_maa (SEQ ID NO:1) which is capable of glutaconyl-CoA reduction to 5-hydroxypent-3-enoate; and (3) the wild type or an engineered variant of E. coli K12 gene pheA (UniProt P0A9J8) encoding prephenate dehydratase (EC 4.2.1.51) which is capable of dehydrating and decarboxylating 5-hydroxypent-3-enoate to 1,3-butadiene. Before synthesis, the genes that are not from E. coli are optimized with a codon bias for expression in E. coli. The synthesized polynucleotides encoding the genes are ligated into an E. coli vector pCK110900 under the control of a lac promoter (as described in International patent publication WO 2011/008535).

The resulting plasmid containing the genes is used to transform E. coli strain K12 using routine transformation methods. Transformed E. coli cells are pre-cultured in LB medium (Difco) with 0.4% glucose and 30 μg/ml chloramphenicol, incubated at 37° C. and 200 rpm with a 2″ throw for 18 hours. Growth is monitored by measuring the optical density at 600 nm. Fresh LB liquid medium including 0.2% glucose and 30 μg/ml chloramphenicol is inoculated with sufficient cells from the pre-culture to obtain a starting optical density of 0.1. After approximately 2 to 3 hours of growth at 37° C. and 250 rpm with a 2″ throw, an optical density of approximately 0.6 is obtained. Isopropylthioglycoside (IPTG) is added to the cells to a final concentration of 1 mM and the cells are incubated at 30° C. and 200 rpm with a 2″ throw for 30-90 minutes until an OD of approximately 1.2 is obtained. Glucose is added to the cells to a final concentration of 2%, the containers are sealed and 1,3-butadiene production is monitored using GC-FID (Agilent GC-GasPro column, 1 ml head space injection, split 10; Method-203° C. for 2.5 min. 250° C. for 2.5 min (ramp 50° C./min), 203%0 for 2 min) with butadiene eluting at 1.9 minutes.

The resulting recombinant host cell comprises an engineered pathway of FIG. 1, Steps A, B, and E and is able to convert crotonyl-CoA to 1,3-butadiene. As described in Example 3, the recombinant E. coli host cell can be grown up in a bioreactor containing a medium comprising the fermentable sugar glucose and produces the 1,3-butadiene product, which is a gas, into the head-space above fermentation medium.

### Example 2

### Recombinant Host Cell with an Engineered Pathway for Production of 1,3-Butadiene Via Phosphate Elimination of a 5-(Phosphonatooxy)pent-3-enoate Intermediate

This Example illustrates the preparation of a recombinant E. coli host cell that expresses the genes in the engineered pathway of FIG. 2 for the production of 1,3-butadiene from fermentable sugar in a fully biosynthetic process.

The following genes of the engineered pathway of FIG. 2, Steps A, B, E, and F are synthesized: (1) the wild type or an engineered variant of Glycine max gene MCCA (Uniprot Q42777) encoding 3-methylcrotonyl-CoA carboxylase (EC 6.4.1.4) which is capable of converting crotonyl-CoA to glutaconyl-CoA: (2) an engineered variant of FAR enzyme (EC 1.1.1*) derived from Marinobacter algicola DG893 gene FAR_maa (SEQ ID NO:1) which is capable of glutaconyl-CoA reduction to 5-hydroxypent-3-enoate; (3) an engineered variant of kinase (EC 2.7.1.x) from S. cerevisiae gene ERG12 (Uniprot P07277) which is capable phosphorylating 5-hydroxypent-3-enoate to 5-(phosphonatooxy)pent-3-enoate; and (4) an engineered variant of a mevalonate diphosphate decarboxylase (EC 4.1.1.x) derived from S. cerevisiae gene MVD (Uniprot P32377), which is capable of phosphate elimination of 5-(phosphonatooxy)pent-3-enoate to produce 1,3-butadiene. Before synthesis, the genes that are not from E. coli are optimized with a codon bias for expression in E. coli. The synthesized polynucleotides encoding the genes are ligated into an E. coli vector pCK110900 under the control of a lac promoter (as described in International patent publication WO 2011/008535).

The resulting plasmid containing the genes is used to transform E. coli strain K12 using routine transformation methods. Transformed E. coli cells are pre-cultured in LB medium (Difco) with 0.4% glucose and 30 μg/ml chloramphenicol, incubated at 37° C. and 200 rpm with a 2″ throw for 18 hours. Growth is monitored by measuring the optical density at 600 nm. Fresh LB liquid medium including 0.2% glucose and 30 μg/ml chloramphenicol is inoculated with sufficient cells from the pre-culture to obtain a starting optical density of 0.1. After approximately 2 to 3 hours of growth at 37° C. and 250 rpm with a 2″ throw, an optical density of approximately 0.6 is obtained. Isopropylthioglycoside (IPTG) is added to the cells to a final concentration of 1 mM and the cells are incubated at 30° C. and 200 rpm with a 2″ throw for 30-90 minutes until an OD of approximately 1.2 is obtained. Glucose is added to the cells to a final concentration of 2%, the containers are sealed and 1,3-butadiene production is monitored using GC-FID (Agilent GC-GasPro column, 1 ml head space injection, split 10; Method-203° C. for 2.5 min, 250° C. for 2.5 min (ramp 50° C./min), 203° C. for 2 min) with butadiene eluting at 1.9 minutes.

The resulting recombinant host cell comprises an engineered pathway of FIG. 2, Steps A. B, E, and F, and is able to convert crotonyl-CoA to 1,3-butadiene. As described in Example 3, the recombinant E. coli host cell can be grown up in a bioreactor containing a medium comprising the fermentable sugar glucose and produces the 1,3-butadiene product, which is a gas, into the head-space above fermentation medium.

### Example 3

### Production and Isolation of 1,3-Butadiene Produced by a Recombinant E. coli Host Cell

This Example illustrates methods and conditions for the large scale production of 1,3-butadiene using a recombinant E. coli host cell of either Example 1 or Example 2 comprising an engineered pathway of FIG. 1 or FIG. 2.

The E. coli host cell is cultured in a fermenter, either in a batch or continuous mode, using a medium containing a fermentable sugar, such as glucose, that is known to support growth of the host cell under anaerobic, aerobic or microaerobic conditions. The expression of the genes encoding the enzymes in the engineered pathways of FIG. 1 or FIG. 2 are induced after the prescribed cell density is reached. Alternatively, a constitutive promoter is used and no induction is necessary. The desired product 1,3-butadiene is a gas under the conditions used in the fermentation, and the amount of 1,3-butadiene produced is monitored by GC sampling of the off-gas from the bioreactor (as generally described in Examples 1 and 2).

The 1,3-butadiene is isolated by directing the fermentation off-gas using a gentle nitrogen sweep, first through a chilled scrubber at 0° C. to condense by-products, primarily water vapor, and then to a cryogenic condenser/trap at −20° C. to collect the 1,3-butadiene as a liquid. The remaining by-product gases, primarily nitrogen and CO₂, then are vented into the atmosphere.

### Example 4

### Optimization of a Recombinant E. coli Host Cell to Increase 1,3-Butadiene Production

This Example illustrates how a recombinant E. coli host cell of Example 1 or 2 comprising an engineered pathway of FIG. 1 or FIG. 2, which is capable of fermenting sugars to produce 1,3-butadiene can be further optimized to increase the productivity (titer and yield) of the desired product.

Briefly, the engineered strain is analyzed as to determine which recombinant gene's expression and/or which enzyme's activity is limiting the production of 1,3-butadiene. A limiting gene's expression can be increased by increasing the copy number in the host cell. If enzyme activity is limiting, it can also be increased by increased copy number of the gene encoding it. Alternatively, the enzyme's gene is engineered via directed evolution to provide a gene encoding an enzyme having increased activity and the host cell is transformed with that recombinant gene. This general process of identifying the limiting gene and/or enzyme followed by increasing copy number and/or enzyme engineering is iterated until the desired amount of production is achieved from the E. coli host cell.

Additionally, metabolic modeling (Biotechnol. Bioengin 2003, 84, 647-657) is utilized to optimize the recombinant E. coli host cell's growth conditions and to knock out genes in the recombinant host cell that are responsible for metabolic leakage/inefficiencies in the engineered pathways of FIG. 1 and FIG. 2. Also, adaptive evolution is used to further optimize production by increasing recombinant host cell's tolerance to inhibitors (see e.g. Science 314, 1565-1568 (2006)).

Each publication, patent, patent application, or other document cited in this application is hereby incorporated by reference in its entirety for all purposes to the same extent as if each were individually indicated to be incorporated by reference for all purposes in the specification directly adjacent the citation.

While various specific embodiments have been illustrated and described, it will be appreciated that various changes can be made without departing from the spirit and scope of the invention(s).

1. A recombinant host cell capable of producing 1,3-butadiene, the host cell comprising:
(a) a recombinant polynucleotide encoding an enzyme capable of converting crotonyl-CoA (or -ACP) to glutaconyl-CoA (or -ACP); and
(b) a recombinant polynucleotide encoding an enzyme capable of converting glutaconyl-CoA (or -ACP) to 5-hydroxypent-3-enoate.

2. The recombinant host cell of claim 1, wherein the host cell further comprises:
(c) a recombinant polynucleotide encoding an enzyme capable of converting 5-hydroxypent-3-enoate to 1,3-butadiene.

3. The recombinant host cell of claim 1, wherein the host cell further comprises:
(c) one or more recombinant polynucleotides encoding an enzyme capable of converting 5-hydroxypent-3-enoate to 5-(phosphonatooxy)pent-3-enoate; and
(d) an enzyme capable of converting 5-(phosphonatooxy)pent-3-enoate to 1,3-butadiene.

4. The recombinant host cell of claim 1, wherein the enzyme capable of converting glutaconyl-CoA (or -ACP) to 5-hydroxypent-3-enoate is a FAR enzyme.

5. The recombinant host cell of claim 4, wherein the recombinant polynucleotide encoding the FAR enzyme comprises one or more nucleotide sequence differences relative to the corresponding naturally occurring polynucleotide, which result in an improved property selected from:
(a) increased activity of the FAR enzyme in the conversion of glutaconyl-CoA (or -ACP) to 5-hydroxypent-3-enoate;
(b) increased expression of the FAR enzyme;
(c) increased host cell tolerance of crotonyl-CoA (or -ACP), glutaconyl-CoA (or -ACP), 5-hydroxypent-3-enoate, 5-(phosphonatooxy)pent-3-enoate, or 1,3-butadiene; or
(d) altered host cell concentration of crotonyl-CoA (or -ACP), glutaconyl-CoA (or -ACP), 5-hydroxypent-3-enoate, 5-(phosphonatooxy)pent-3-enoate, or 1,3-butadiene.

6. The recombinant host cell of claim 4, wherein the recombinant polynucleotide encoding a FAR enzyme comprises a polynucleotide sequence that has at least 80% identity to, or hybridizes under stringent conditions to, a sequence encoding a FAR enzyme of any one of SEQ ID NO: 1, 2, 3, or 4.

7. The recombinant host cell of claim 4, wherein the FAR enzyme (a) comprises an amino acid sequence having at least 80%, at least 85%, at least 90%, at least 95%, at least 96%, at least 97%, at least 98%, at least 99%, or more, identity to an amino acid sequence of any one of SEQ ID NO: 1, 2, 3, or 4; and/or (b) is an engineered fatty acyl reductase derived from an amino acid sequence of any one of SEQ ID NO: 1, 2, 3, or 4.

8. The recombinant host cell of claim 1, wherein the enzyme capable of converting crotonyl-CoA (or -ACP) to glutaconyl-CoA (or -ACP) is an engineered methylcrotonyl-CoA carboxylase or a geranoyl-CoA carboxylase derived from any one of the following enzymes:

| | | | | GI |
|---|---|---|---|---|
| Gene | Organism | UniProt id | GenBank id | Number |
| Mccc1 | Mus musculus | Q99MR8 | AF313338.1 | 12276064 |
| Mccc2 | Mus musculus | Q3ULD5 | AK132265.1 | 74205533 |
| MCCA | Glycine max | Q42777 | AAA53141.1 | 497234 |
| MCCB | Arabidopsis thaliana | Q9LDD8 | AF059511.1 | 7021224 |
| atuF | Pseudomonas | Q9HZV6 | AAG06279.1 | 9948982 |
| | aeruginosa | | | |
| atuC | Pseudomonas | Q9HZV6 | AAG06276.1 | 9948979 |
| | aeruginosa | | | |

9. The recombinant host cell of claim 1, wherein the enzyme capable of converting 5-hydroxypent-3-enoate to 1,3-butadiene is an engineered prephenate dehydratase or arogenate dehydratase derived from any one of the following enzymes:

| Gene | Organism | UniProt id | GenBank id | GI Number |
|---|---|---|---|---|
| ADT1 | Arabidopsis thaliana | Q9SA96 | AAD30242.1 | 4835776 |
| ADT2 | Arabidopsis thaliana | Q9SSE7 | AEE74577.1 | 332641056 |
| ADT3 | Arabidopsis thaliana | Q9ZUY3 | AEC08050.1 | 330252956 |
| ADT4 | Arabidopsis thaliana | O2241 | AEE77939.1 | 332644418 |
| ADT5 | Arabidopsis thaliana | Q9FNJ8 | AED93055.1 | 332005672 |
| ADT6 | Arabidopsis thaliana | Q9SGD6 | AEE28265.1 | 332190144 |
| pheA | Escherichia coli | P0A9J9 | AAG57710.1 | 12517021 |
| | O157:H7 | | | |
| pheA | Escherichia coli K12 | P0A9J8 | AAA24330.1 | 147175 |
| pheA | Methanocaldococcus jannaschii | Q58054 | AAB98631.1 | 1591349 |
| pheC | Pseudomonas aeruginosa | Q01269 | AAC08596.1 | 2997758 |

10. The recombinant host cell of claim 1, wherein the enzyme capable of converting 5-hydroxypent-3-enoate to 5-(phosphonatooxy)pent-3-enoate is an engineered alcohol kinase derived from any one of the following enzymes:

| | | | | GI |
|---|---|---|---|---|
| Gene | Organism | UniProt id | GenBank id | Number |
| GUT1 | Saccharomyces | P32190 | CAA48791.1 | 312423 |
| | cerevisiae | | | |
| glpK | Escherichia coli | P0A6F3 | AAA23913.1 | 142660 |
| | (strain K12) | | | |
| CHKA | Homo sapiens | P35790 | BAA01547.1 | 219541 |
| Chka | Mus musculus | O54804 | BAA88153.1 | 6539495 |
| Chkb | Mus musculus | O55229 | BAA24891.1 | 2897731 |
| ckb-2 | Caenorhabditis | P46559 | CAA84301.2 | 29603337 |
| | elegans | | | |
| CKI1 | Saccharomyces | P20485 | AAA34499.1 | 171231 |
| | cerevisiae | | | |
| MVK | Homo sapiens | Q03426 | AAF82407.1 | 9049533 |
| mvk | Dictyostelium | Q86AG7 | EAL71443.1 | 60472399 |
| | discoideum | | | |
| mvk | Methanocaldococcus | Q58487 | AAB99088.1 | 1591731 |
| | jannaschii | | | |
| Mvk | Rattus norvegicus | P17256 | AAA41588.1 | 205378 |
| ERG12 | Saccharomyces | P07277 | CAA39359.1 | 3684 |
| | cerevisiae | | | |
| mk | Arabidopsis thaliana | P46086 | AAD31719.1 | 4883990 |
| THR1 | Saccharomyces | P17423 | AAA34154.1 | 172978 |
| | cerevisiae | | | |
| thrB | Escherichia coli | P00547 | AAA50618.1 | 529240 |
| | (strain K12) | | | |
| thrB | Methanocaldococcus | Q58504 | AAB99107 | 1591748 |
| | jannaschii | | | |

11. The recombinant host cell of claim 1, wherein the enzyme capable of converting 5-(phosphonatooxy)pent-3-enoate to 1,3-butadiene is an engineered diphosphomevalonate decarboxylase derived from any one of the following enzymes:

| | | | | GI |
|---|---|---|---|---|
| Gene | Organism | UniProt id | GenBank id | Number |
| MVD | Homo sapiens | P53602 | EAW66792.1 | 119587196 |
| MVD1 | Saccharomyces | P32377 | CAA66158 | 1292890 |
| | cerevisiae | | | |
| Mvd | Mus musculus | Q99JFA | CAC35731 | 13539580 |
| mvaD | Streptococcus | Q9A097 | AAK33797.1 | 13622042 |
| | pygenes serotype | | | |
| | M1 | | | |

12. The recombinant host cell of claim 1, wherein the host cell is capable producing 1,3-butadiene by fermentation of a carbon source, optionally a fermentable sugar, optionally obtained from a cellulosic biomass.

13. The recombinant host cell of claim 1, wherein the host cell is from a strain of microorganism derived from any one of: Escherichia coli, Bacillus, Saccharomyces, Streptomyces, and Yarrowia.

14. A method of producing 1,3-butadiene comprising contacting the recombinant host cell of claim 1, a medium comprising a carbon source under suitable conditions for generating 1,3-butadiene, optionally further comprising a step of recovering 1,3-butadiene produced by the recombinant host cell.

15. The method of claim 14, wherein the carbon source comprises a fermentable sugar, optionally obtained from cellulosic biomass.

16. A method of manufacturing a recombinant host cell of claim 1, the method comprising transforming a suitable host cell with one or more nucleic acid constructs encoding:
(a) an enzyme capable of converting crotonyl-CoA (or -ACP) to glutaconyl-CoA (or -ACP);
(b) an enzyme capable of converting glutaconyl-CoA (or -ACP) to 5-hydroxypent-3-enoate;
(c) an enzyme capable of converting 5-hydroxypent-3-enoate to 1,3-butadiene;
(d) an enzyme capable of converting 5-hydroxypent-3-enoate to 5-(phosphonatooxy)pent-3-enoate; and/or
(e) an enzyme capable of converting 5-(phosphonatooxy)pent-3-enoate to 1,3-butadiene.
